(19) United States
(12) Patent Application Publication
(10) Pub. No.: US 2018/0282297 A1
(43) Pub. Date: Oct. 4, 2018

(54) POLYMORPHS OF 3-(4-AMINO-1-OXOISOINDOLIN-2-YL)PIPERIDINE-2,6-DIONE

(71) Applicant: Shenzhen Jingtai Technology Co., Ltd., Shenzhen (CN)

(72) Inventors: Junbo GONG, Tianjin (CN); Lina JIA, Tianjin (CN); Shuhao WEN, Shenzhen (CN); Jian MA, Shenzhen (CN); Jingkang WANG, Tianjin (CN); Qiuxiang YIN, Tianjin (CN); Baohong HOU, Tianjin (CN); Lipeng LAI, Shenzhen (CN); Peiyu ZHANG, Shenzhen (CN); Mingjun YANG, Shenzhen (CN); Yang LIU, Shenzhen (CN); Guangxu SUN, Shenzhen (CN)

(21) Appl. No.: 15/936,788

(22) Filed: Mar. 27, 2018

Related U.S. Application Data
(60) Provisional application No. 62/484,503, filed on Apr. 12, 2017.

(30) Foreign Application Priority Data
Mar. 27, 2017 (CN) 201710189502.7

(51) Int. Cl. C07D 401/04; B01D 9/00; A61P 35/00

(52) U.S. Cl. CPC C07D 401/04; A61P 35/00; B01D 9/005; B01D 9/0045

(57) ABSTRACT

The present application relates to polymorphs of Compound A:

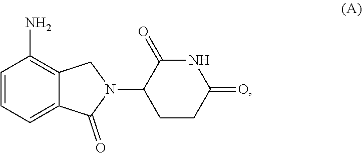 [SMILES: Nc1cccc2c1CN(C1CCC(=O)NC1=O)C2=O]

or a stereoisomer thereof, and methods of preparation and use thereof.

### RELATED APPLICATIONS

This application claims priority to, and the benefit of, Chinese Patent Application No. 201710189502.7, filed on Mar. 27, 2017, and U.S. Application No. 62/484,503, filed on Apr. 12, 2017, the entire contents of each of which are incorporated herein by reference in their entireties.

### BACKGROUND

3-(4-amino-1-oxoisoindolin-2-yl)piperidine-2,6-dione (also known as lenalidomide) has been used in the treatment of various cancers, such as multiple myeloma and non-Hodgkin's lymphoma. Lenalidomide has also shown efficacy in other diseases or conditions, including myelodysplastic syndromes, chronic lymphocytic leukemia, and solid tumors. Lenalidomide possesses anti-neoplastic activity and modulates immunologic effects, including blocking tumor cell proliferation and angiogenesis, and stimulating T-cell and natural killer (NK) cell mediated cytotoxicity.

Polymorphism of a compound affects many of the compound's properties, such as solubility, hygroscopicity, chemical reactivity, and stability. Many of the inconsistencies encountered in drug performance can be attributed to polymorphism. Despite the importance of polymorphism, methods of predicting the existence of possible polymorphs of a compound and conditions under which they can be formed are unreliable, and processes for producing polymorphs often fail to generate them consistently and reliably.

Accordingly, new polymorphs of lenalidomide which display desirable properties (e.g., improved solubility, hygroscopicity, chemical reactivity, and/or stability) are needed to advance the development of the compound as a therapeutic agent. The present application addresses the need.

### SUMMARY

This application pertains, at least in part, to a polymorph of a dihydrate of Compound A:

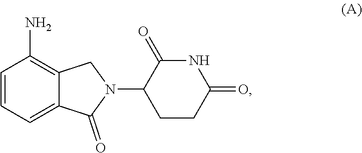 [SMILES: Nc1cccc2c1CN(C1CCC(=O)NC1=O)C2=O]

or a stereoisomer thereof (“Form DH”).

The application also pertains, at least in part, to Form DH, characterized by an X-ray powder diffraction (XRPD) pattern comprising peaks at approximately 12.0, 13.6, and 24.7° 20 using Cu Kα radiation.

The application also pertains, at least in part, to Form DH, characterized by an XRPD pattern substantially similar to that set forth in FIG. 1.

This application pertains, at least in part, to a polymorph of Compound A anhydrate or a stereoisomer thereof (“Form α”).

The application also pertains, at least in part, to Form α, characterized by an XRPD pattern comprising peaks at approximately 17.6, 20.5, and 24.1° 2θ using Cu Kα radiation.

The application also pertains, at least in part, to Form α, characterized by an XRPD pattern substantially similar to that set forth in FIG. 16.

The application also pertains, at least in part, to a pharmaceutical composition comprising a polymorph of the present application, and a pharmaceutically acceptable excipient or carrier.

The application also pertains, at least in part, to a method of preparing a polymorph of the present application.

The application also pertains, at least in part, to a method of treating or preventing a disease or condition in a subject in need thereof, comprising administering to the subject a therapeutically effective amount of a polymorph of the present application.

The application also pertains, at least in part, to a polymorph of the present application for treating or preventing a disease or condition.

The application also pertains, at least in part, to use of a polymorph of the present application in the treatment or prevention of a disease or condition.

The application also pertains, at least in part, to use of a polymorph of the present application in the manufacture of a medicament for treatment or prevention of a disease or condition.

Unless otherwise defined, all technical and scientific terms used herein have the same meaning as commonly understood by one of ordinary skill in the art to which this application belongs. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art. In the case of conflict, the present specification, including definitions, will control. In addition, the materials, methods, and examples are illustrative only and are not intended to be limiting. Other features and advantages of the application will be apparent from the following detailed description and claims.

### BRIEF DESCRIPTION OF THE FIGURES

FIG. 1: A graph showing the XRPD of Form DH

FIG. 2: A graph showing the DVS of Form DH

FIG. 3: A graph showing the XRPD of Form DH after dynamic vapor sorption

FIG. 4: A graph showing the TG of Form DH

FIG. 5: A graph showing the DSC of Form DH

FIG. 6: A graph showing the IR spectrum of Form DH

FIG. 7: A graph showing the Raman spectrum of Form DH

FIG. 8: A graph showing the XRPD of Form DH after stability test in methanol

FIG. 9: A graph showing the XRPD of Form DH after stability test in acetone

FIG. 10: A graph showing the XRPD of Form DH after being heated to 170° C.

FIG. 11: A graph showing the XRPD of Form DH after being heated to 190° C.

FIG. 12: A graph showing the XRPD of the polymorph obtained in Example 2

FIG. 13: A graph showing the TG of the polymorph obtained in Example 2

FIG. 14: A graph showing the DSC of the polymorph obtained in Example 2

FIG. 15: A graph showing the XRPD of Form DH obtained in Example 2

FIG. 16: A graph showing the XRPD of Form α

FIG. 17: A graph showing the DVS of Form α

FIG. 18: A graph showing the XRPD of Form α after dynamic vapor sorption

FIG. 19: A graph showing the TG of Form α

FIG. 20: A graph showing the DSC of Form α

FIG. 21: A graph showing the IR spectrum of Form α

FIG. 22: A graph showing the Raman spectrum of Form α

FIG. 23: A graph showing the ¹H NMR spectrum of Form α

FIG. 24: A graph showing the XRPD of Form α after stability test in methanol

FIG. 25: A graph showing the XRPD of Form α after stability test in water

FIG. 26: A graph showing the XRPD of Form α after heat conversion

FIG. 27: A graph showing the XRPD of Form DH after 28 days at 40° C./75% RH

FIG. 28: A graph showing the XRPD of Form DH after 28 days at RT/P₂O₅

FIG. 29: A graph showing the XRPD of Form α after 28 days at 40° C./75% RH

FIG. 30: A graph showing the XRPD of Form α after 28 days at RT/P₂O₅

FIG. 31: A graph showing powder dissolution curve of the polymorphs as indicated

FIG. 32: A graph showing a standard UV curve of Compound A in water

### DETAILED DESCRIPTION

### Polymorphs of the Application

### Form DH

The application pertains, at least in part, to a polymorph of a dihydrate of Compound A:

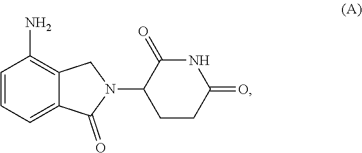 [SMILES: Nc1cccc2c1CN(C1CCC(=O)NC1=O)C2=O]

or a stereoisomer thereof (“Form DH”).

In one embodiment, Form DH is characterized by an XRPD pattern comprising peaks at approximately 12.0, 13.6, and 24.7° 2θ using Cu Kα radiation.

In one embodiment, Form DH is characterized by an XRPD pattern comprising peaks at 11.960±0.2, 13.619±0.2, and 24.660±0.2° 2θ using Cu Kα radiation.

In one embodiment, Form DH is characterized by an XRPD pattern comprising peaks at approximately 12.0, 13.6, 24.7, and 27.5° 2θ using Cu Kα radiation.

In one embodiment, Form DH is characterized by an XRPD pattern comprising peaks at 11.960±0.2, 13.619±0.2, 24.660±0.2, and 27.480±0.2° 2θ using Cu Kα radiation.

In one embodiment, Form DH is characterized by an XRPD pattern comprising peaks at approximately 12.0, 13.6, 24.1, 24.7, 25.4, and 27.5° 2θ using Cu Kα radiation.

In one embodiment, Form DH is characterized by an XRPD pattern comprising peaks at 11.960±0.2, 13.619±0.2, 24.100±0.2, 24.660±0.2, 25.400±0.2, and 27.480±0.2° 2θ using Cu Kα radiation.

In one embodiment, Form DH is characterized by an XRPD pattern comprising peaks at approximately 12.0, 13.6, 20.1, 22.7, 24.1, 24.7, 25.4, 26.7, 27.5, and 28.7° 2θ using Cu Kα radiation.

In one embodiment, Form DH is characterized by an XRPD pattern comprising peaks at 11.960±0.2, 13.619±0.2, 20.059±0.2, 22.680±0.2, 24.100±0.2, 24.660±0.2, 25.400±0.2, 26.738±0.2, 27.480±0.2, and 28.661±0.2° 2θ using Cu Kα radiation.

In one embodiment, Form DH is characterized by an XRPD pattern comprising peaks at approximately 12.0, 13.6, 15.2, 18.6, 20.1, 21.2, 21.4, 22.1, 22.7, 23.3, 24.1, 24.7, 25.4, 26.7, 27.5, and 28.7° 2θ using Cu Kα radiation.

In one embodiment, Form DH is characterized by an XRPD pattern comprising peaks at 11.960±0.2, 13.619±0.2, 15.220±0.2, 18.621±0.2, 20.059±0.2, 21.161±0.2, 21.436±0.2, 22.100±0.2, 22.680±0.2, 23.259±0.2, 24.100±0.2, 24.660±0.2, 25.400±0.2, 26.738±0.2, 27.480±0.2, and 28.661±0.2° 2θ using Cu Kα radiation.

In one embodiment, Form DH is characterized by an XRPD pattern comprising peaks at approximately 12.0, 12.5, 13.6, 15.2, 18.6, 20.1, 21.2, 21.4, 22.1, 22.7, 23.3, 24.1, 24.7, 25.4, 26.7, 27.5, 28.7, and 29.9° 2θ using Cu Kα radiation.

In one embodiment, Form DH is characterized by an XRPD pattern comprising peaks at 11.960±0.2, 12.538±0.2, 13.619±0.2, 15.220±0.2, 18.621±0.2, 20.059±0.2, 21.161±0.2, 21.436±0.2, 22.100±0.2, 22.680±0.2, 23.259±0.2, 24.100±0.2, 24.660±0.2, 25.400±0.2, 26.738±0.2, 27.480±0.2, 28.661±0.2, and 29.885±0.2° 2θ using Cu Kα radiation.

In one embodiment, Form DH is characterized by an XRPD pattern comprising peaks at approximately the positions shown in the table below:

| Peak No. | 2-Theta | d-(A) | Intensity | L/L0 |
|---|---|---|---|---|
| 1 | 11.960 | 7.3937 | 922 | 55.7 |
| 2 | 12.538 | 7.0542 | 94 | 5.7 |
| 3 | 13.619 | 6.4963 | 1357 | 82 |
| 4 | 15.220 | 5.8165 | 177 | 10.7 |
| 5 | 17.439 | 5.0812 | 90 | 5.4 |
| 6 | 18.259 | 4.8546 | 38 | 2.3 |
| 7 | 18.621 | 4.7612 | 149 | 9 |
| 8 | 20.059 | 4.423 | 248 | 15 |
| 9 | 21.161 | 4.1951 | 132 | 8 |
| 10 | 21.436 | 4.1418 | 147 | 8.9 |
| 11 | 22.100 | 4.0189 | 151 | 9.1 |
| 12 | 22.680 | 3.9173 | 342 | 20.7 |
| 13 | 23.259 | 3.8211 | 182 | 11 |
| 14 | 24.100 | 3.6897 | 538 | 32.5 |
| 15 | 24.660 | 3.6071 | 1654 | 100 |
| 16 | 25.400 | 3.5037 | 628 | 38 |
| 17 | 26.738 | 3.3314 | 278 | 16.8 |
| 18 | 27.480 | 3.2431 | 758 | 45.8 |
| 19 | 28.661 | 3.112 | 251 | 15.2 |
| 20 | 29.885 | 2.9873 | 126 | 7.6 |
| 21 | 30.500 | 2.9285 | 77 | 4.7 |
| 22 | 32.003 | 2.7943 | 179 | 10.8 |
| 23 | 33.320 | 2.6868 | 46 | 2.8 |
| 24 | 33.621 | 2.6634 | 54 | 3.3 |
| 25 | 34.220 | 2.6182 | 104 | 6.3 |
| 26 | 34.679 | 2.5845 | 173 | 10.5 |
| 27 | 35.662 | 2.5155 | 45 | 2.7 |
| 28 | 36.655 | 2.4497 | 31 | 1.9 |
| 29 | 38.279 | 2.3493 | 57 | 3.4 |
| 30 | 39.020 | 2.3064 | 61 | 3.7 |
| 31 | 39.501 | 2.2794 | 71 | 4.3 |

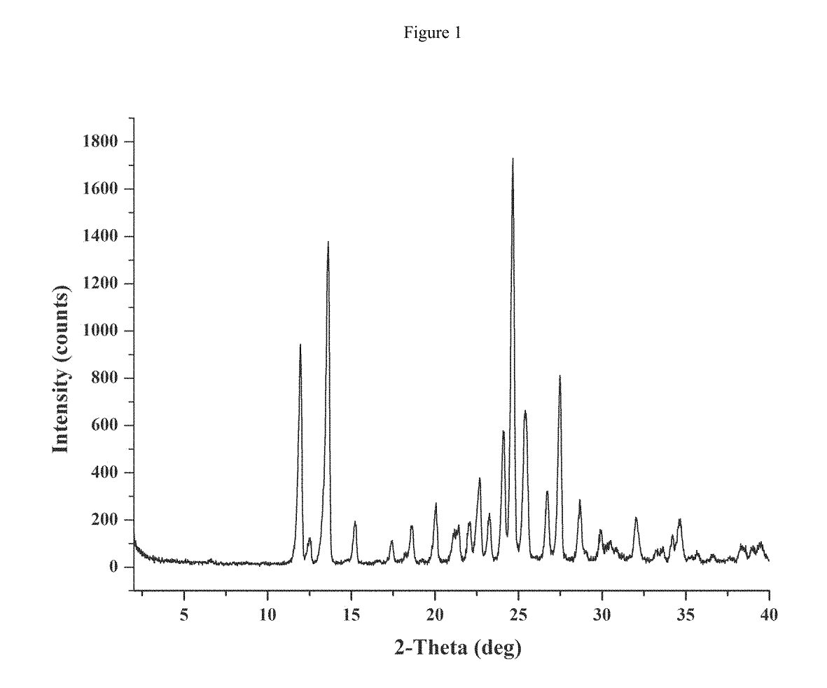

In one embodiment, Form DH is characterized by an XRPD pattern substantially similar to that set forth in FIG. 1.

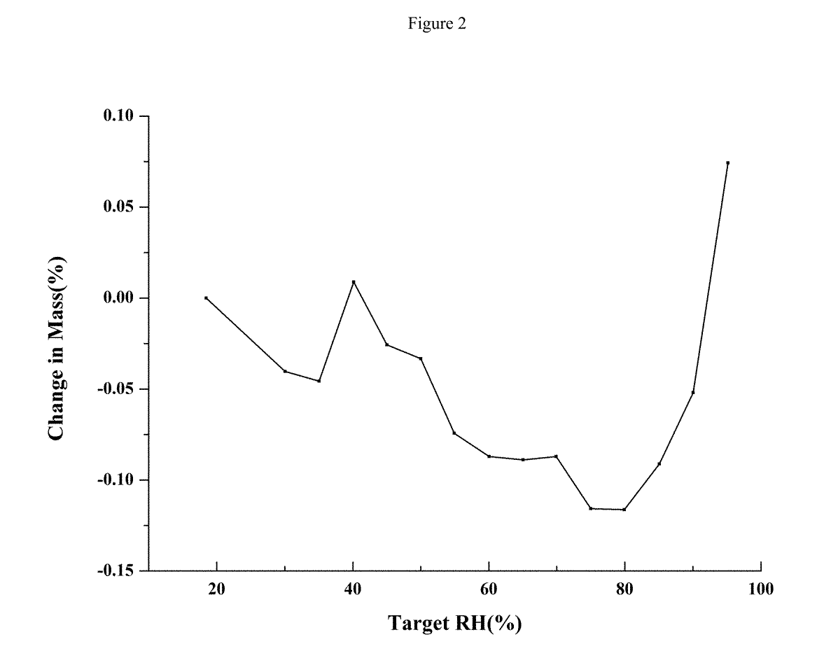

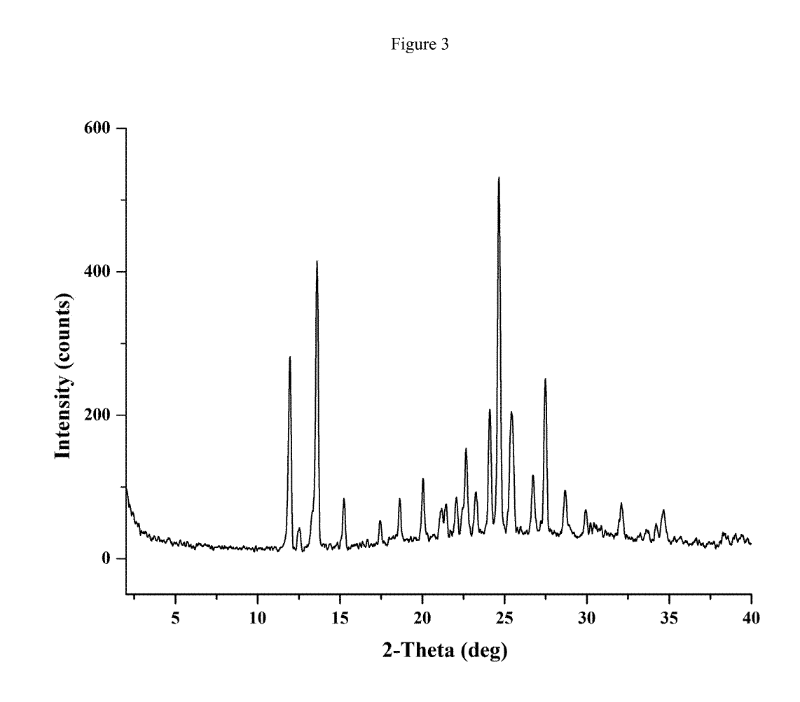

In one embodiment, Form DH is stable under various humidity. In one embodiment, Form DH displays low hygroscopicity under various humidity. In one embodiment, the water content of Form DH does not change significantly (e.g., less than 0.01%, 0.02%, 0.03%, 0.04%, 0.05%, 0.06%, 0.07%, 0.08%, 0.09%, or 0.1%), upon change in humidity. In one embodiment, the water content of Form DH does not change significantly at a humidity between 20% RH and 95% RH. In one embodiment, the change of the water content of Form DH is as set forth in FIG. 2. In one embodiment, the XRPD pattern of Form DH after exposure to various humidity (e.g., between 20% RH and 95% RH) is substantially the same as the XRPD pattern of Form DH before exposure to humidity. In one embodiment, the XRPD pattern of Form DH after exposure to various humidity (e.g., between 20% RH and 95% RH) is substantially similar to that set forth in FIG. 3.

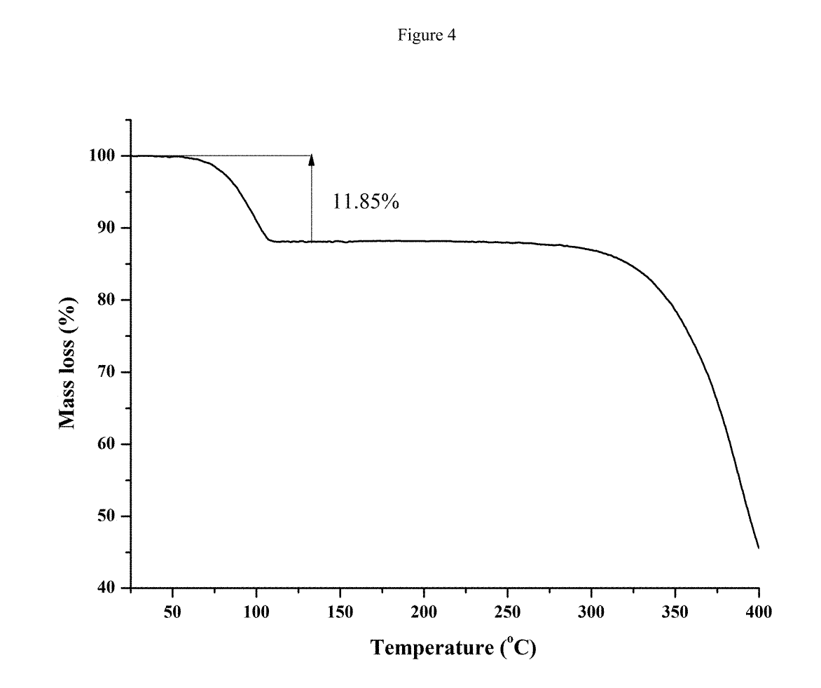

In one embodiment, Form DH shows weight loss of approximately between 10% and 15% when heated to approximately 110° C., as measured by TGA. In one embodiment, Form DH shows weight loss of approximately 12.5% when heated to approximately 110° C., as measured by TGA. In one embodiment, Form DH shows weight loss of 12.2±0.5% when heated to approximately 110° C., as measured by TGA. In one embodiment, Form DH is characterized by a TGA pattern substantially similar to that set forth in FIG. 4.

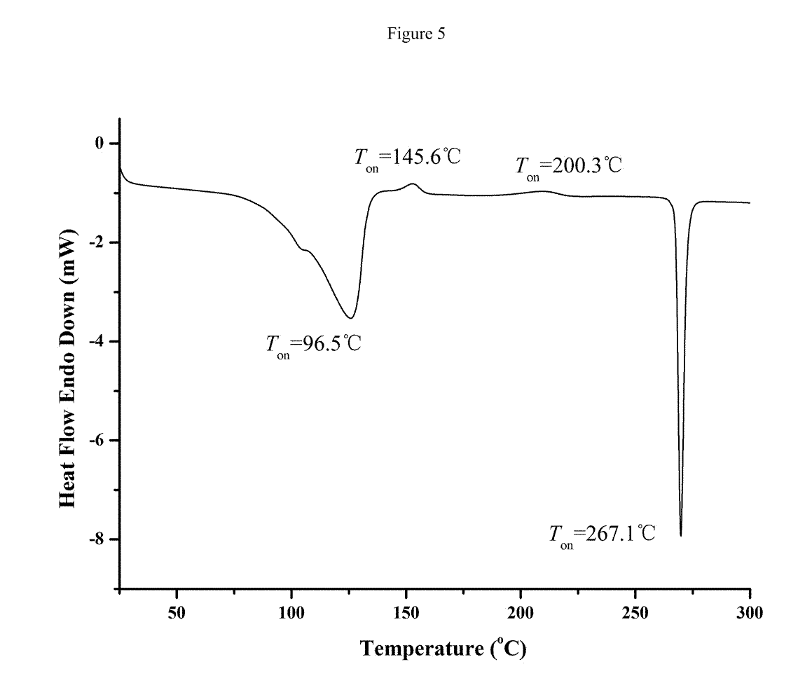

In one embodiment, Form DH is characterized by an endothermic event with onset at approximately 96° C., as measured by DSC. In one embodiment, Form DH is characterized by an endothermic event with onset at 96.5±5° C., as measured by DSC. In one embodiment, Form DH is characterized by an exothermic event with onset at approximately 146° C. and/or 200° C., as measured by DSC. In one embodiment, Form DH is characterized by an exothermic event with onset at 145.6±5° C. and/or 200.3±5° C., as measured by DSC. In one embodiment, Form DH is characterized by a melting event with onset at approximately 267° C., as measured by DSC. In one embodiment, Form DH is characterized by a melting event with onset at 267.1±5° C., as measured by DSC. In one embodiment, Form DH is characterized by a DSC thermogram substantially similar to that set forth in FIG. 5.

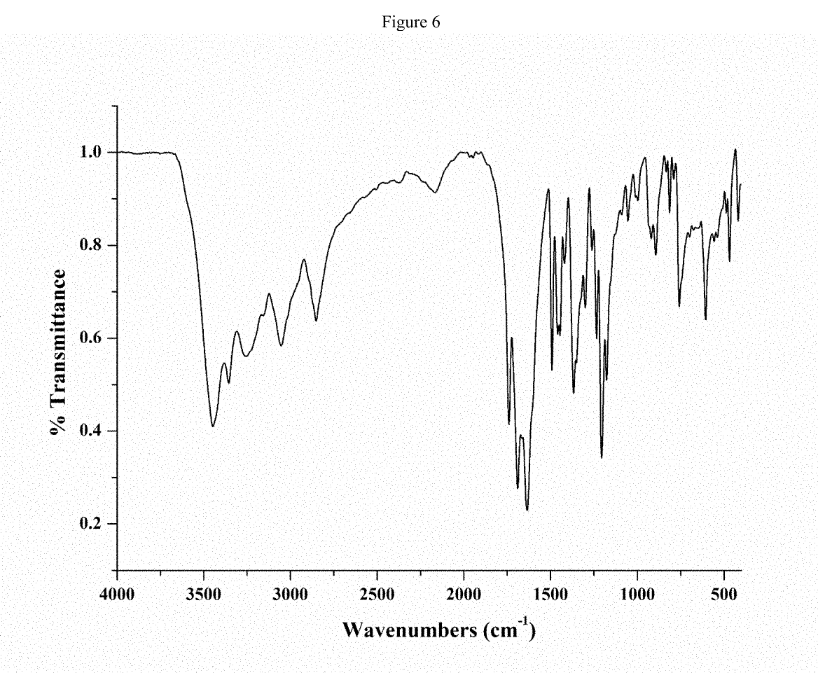

In one embodiment, Form DH is characterized by an IR spectrum comprising peaks at approximately 3447, 3356, 3256, 3053, 2852, 1740, 1690, 1635, 1206, 759, and 607 cm′. In one embodiment, Form DH is characterized by an IR spectrum substantially similar to that set forth in FIG. 6.

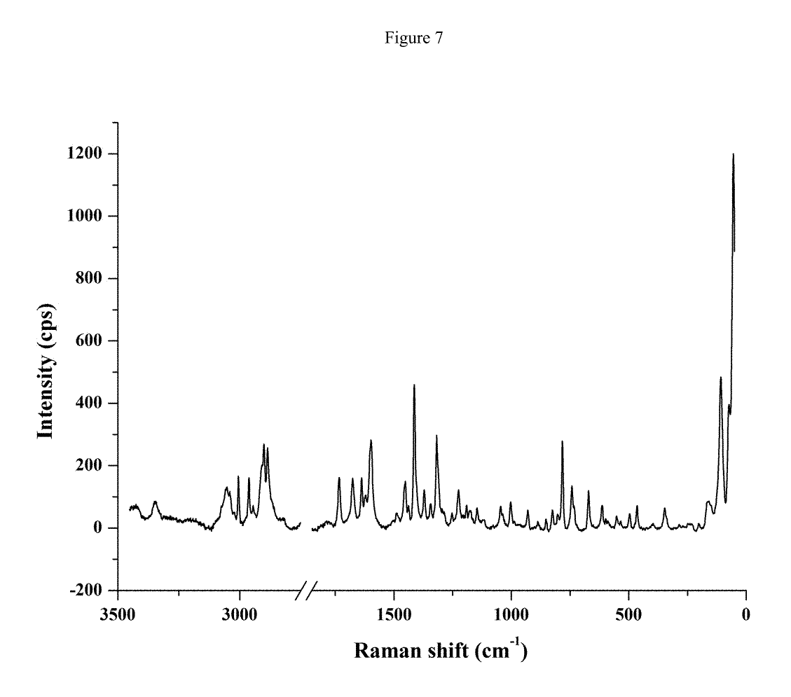

In one embodiment, Form DH is characterized by a Raman spectrum comprising peaks at approximately 2901, 2887, 1598, 1414, 1318, and 783 cm⁻¹. In one embodiment, Form DH is characterized by a Raman spectrum substantially similar to that set forth in FIG. 7.

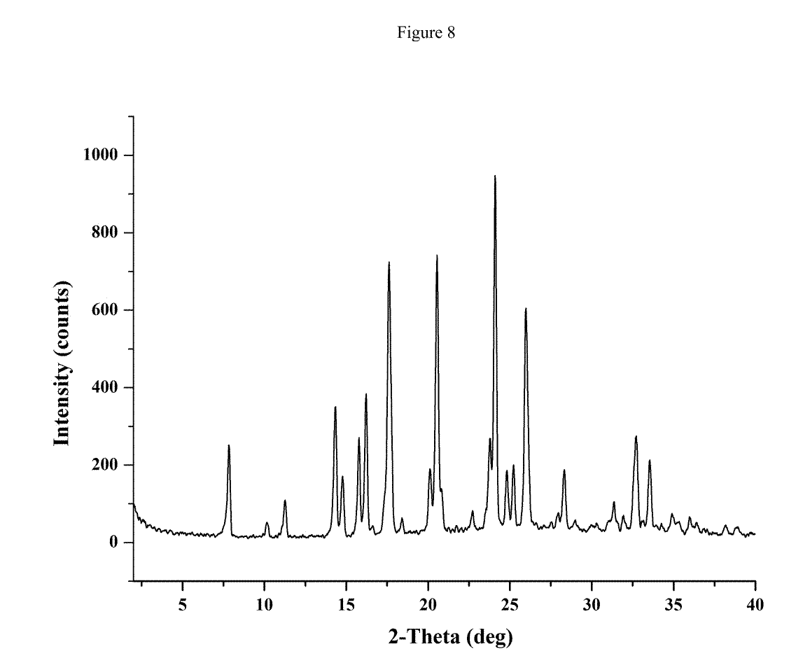

In one embodiment, Form DH is stable upon mixing with a variety of solvents. In one embodiment, the solvent is selected from water, methanol, ethanol, acetonitrile, tetrahydrofuran, ethyl acetate, and dioxane. In one embodiment, upon mixing with methanol, Form DH maintains its polymorphic form as Form DH. In one embodiment, upon mixing with methanol, Form DH is characterized by an XRPD pattern substantially similar to that set forth in FIG. 8.

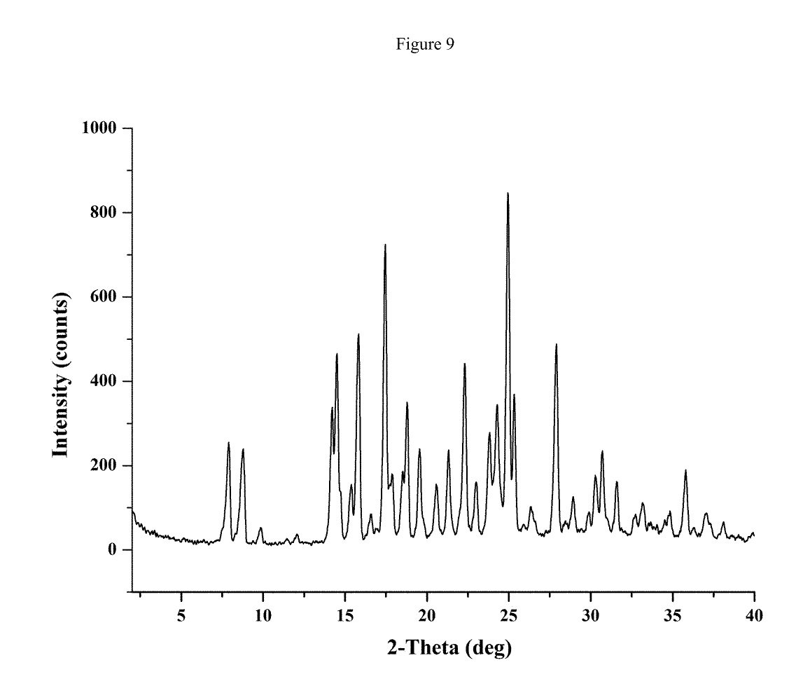

In one embodiment, upon mixing with acetone, Form DH converts to a polymorph characterized by an XRPD pattern substantially similar to that set forth in FIG. 9 (“Form C”).

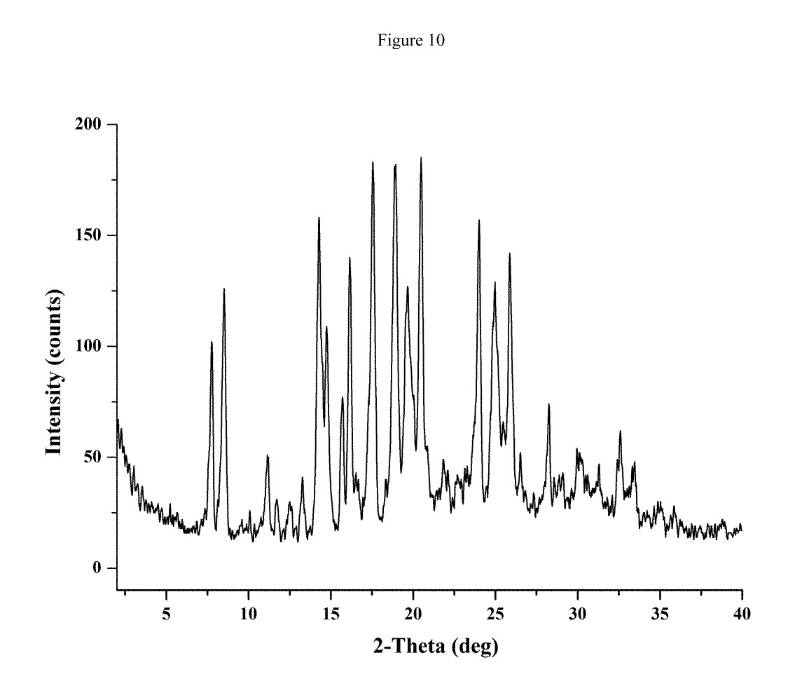

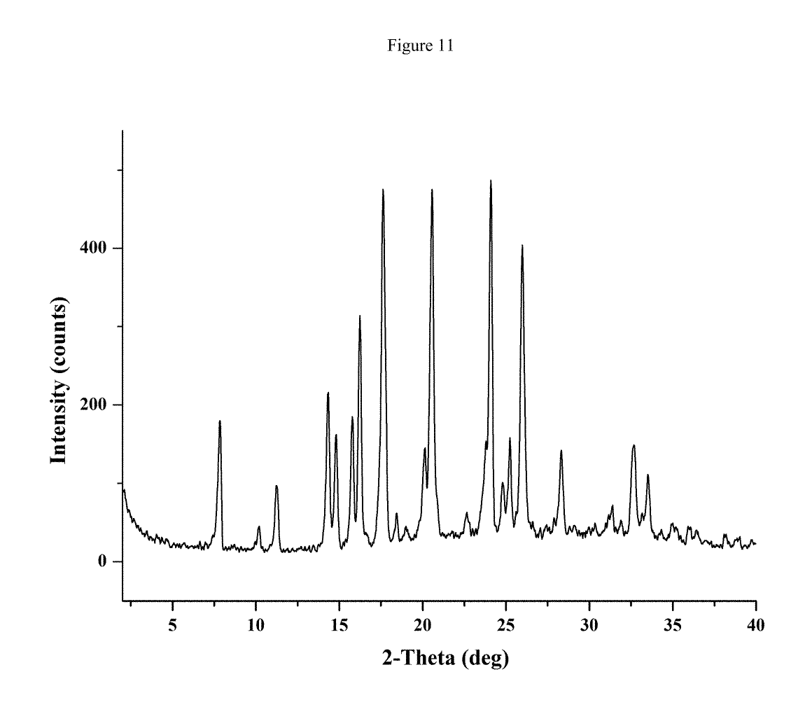

In one embodiment, Form DH converts to a different polymorph at an elevated temperature (e.g., 170° C. or higher). In one embodiment, when heated to 170° C., Form DH converts to a polymorph characterized by an XRPD pattern substantially similar to that set forth in FIG. 10 (“Form F”). In one embodiment, when heated to 190° C., Form DH converts to a polymorph characterized by an XRPD pattern substantially similar to that set forth in FIG. 11 (“Form A”).

In one embodiment, Form DH displays high crystallinity (e.g., higher crystallinity as compared to other polymorphs or crystal forms of Compound A), and/or high stability (e.g., higher stability as compared to other polymorphs or crystal forms of Compound A) when Form DH and the other polymorphs are subjected to substantially the same storage conditions (e.g., humidity and/or temperature).

In one embodiment, Form DH is stable under various storage conditions. In one embodiment, Form DH is stable (e.g., the XRPD pattern of Form DH does not substantially change) after storage under 20° C./75% RH, 25° C./75% RH, 30° C./75% RH, 35° C./75% RH, 40° C./75% RH, 50° C./75% RH, 20° C./90% RH, 25° C./90% RH, 30° C./90% RH, 35° C./90% RH, 40° C./90% RH, 50° C./90% RH, 20° C./95% RH, 25° C./95% RH, 30° C./95% RH, 35° C./95% RH, 40° C./95% RH, or 50° C./95% RH, for at least a week, two weeks, three weeks, four weeks, six weeks, eight weeks, three months, four months, five months, six months, eight months, ten months, or twelve months. In one embodiment, Form DH is stable after storage under 40° C./75% RH for at least four weeks.

### Form α

The application pertains, at least in part, to a polymorph of Compound A anhydrate or a stereoisomer thereof (“Form α”).

In one embodiment, Form α is characterized by an XRPD pattern comprising peaks at approximately 17.6, 20.5, and 24.1° 2θ using Cu Kα radiation.

In one embodiment, Form α is characterized by an XRPD pattern comprising peaks at 17.601±0.2, 20.500±0.2, and 24.061±0.2° 2θ using Cu Kα radiation.

In one embodiment, Form α is characterized by an XRPD pattern comprising peaks at approximately 17.6, 20.5, 24.1, and 26.0° 2θ using Cu Kα radiation.

In one embodiment, Form α is characterized by an XRPD pattern comprising peaks at 17.601±0.2, 20.500±0.2, 24.061±0.2, and 25.980±0.2° 2θ using Cu Kα radiation.

In one embodiment, Form α is characterized by an XRPD pattern comprising peaks at approximately 16.2, 17.6, 20.5, 24.1, and 26.0° 2θ using Cu Kα radiation.

In one embodiment, Form α is characterized by an XRPD pattern comprising peaks at 16.218±0.2, 17.601±0.2, 20.500±0.2, 24.061±0.2, and 25.980±0.2° 2θ using Cu Kα radiation.

In one embodiment, Form α is characterized by an XRPD pattern comprising peaks at approximately 7.8, 14.3, 15.8, 16.2, 17.6, 20.5, 24.1, and 26.0° 2θ using Cu Kα radiation. In one embodiment, Form α is characterized by an XRPD pattern comprising peaks at 7.780±0.2, 14.302±0.2, 15.760±0.2, 16.218±0.2, 17.601±0.2, 20.500±0.2, 24.061±0.2, and 25.980±0.2° 2θ using Cu Kα radiation.

In one embodiment, Form α is characterized by an XRPD pattern comprising peaks at approximately 7.8, 14.3, 14.7, 15.8, 16.2, 17.6, 20.1, 20.5, 24.1, 25.2, 26.0, 28.3, 32.6, and 33.5° 2θ using Cu Kα radiation.

In one embodiment, Form α is characterized by an XRPD pattern comprising peaks at 7.780±0.2, 14.302±0.2, 14.721±0.2, 15.760±0.2, 16.218±0.2, 17.601±0.2, 20.122±0.2, 20.500±0.2, 24.061±0.2, 25.199±0.2, 25.980±0.2, 28.263±0.2, 32.639±0.2, and 33.540±0.2° 2θ using Cu Kα radiation.

In one embodiment, Form α is characterized by an XRPD pattern comprising peaks at approximately 7.8, 8.2, 11.3, 14.3, 14.7, 15.8, 16.2, 17.6, 20.1, 20.5, 24.1, 24.8, 25.2, 26.0, 28.3, 32.6, and 33.5° 2θ using Cu Kα radiation.

In one embodiment, Form α is characterized by an XRPD pattern comprising peaks at 7.780±0.2, 8.220±0.2, 11.279±0.2, 14.302±0.2, 14.721±0.2, 15.760±0.2, 16.218±0.2, 17.601±0.2, 20.122±0.2, 20.500±0.2, 24.061±0.2, 24.816±0.2, 25.199±0.2, 25.980±0.2, 28.263±0.2, 32.639±0.2, and 33.540±0.2° 2θ using Cu Kα radiation.

In one embodiment, Form α is characterized by an XRPD pattern comprising peaks at approximately 7.8, 8.2, 10.2, 11.3, 14.3, 14.7, 15.8, 16.2, 17.6, 18.4, 20.1, 20.5, 24.1, 24.8, 25.2, 26.0, 28.3, 31.3, 32.6, 33.5, and 35.0° 2θ using Cu Kα radiation.

In one embodiment, Form α is characterized by an XRPD pattern comprising peaks at 7.780±0.2, 8.220±0.2, 10.218±0.2, 11.279±0.2, 14.302±0.2, 14.721±0.2, 15.760±0.2, 16.218±0.2, 17.601±0.2, 18.402±0.2, 20.122±0.2, 20.500±0.2, 24.061±0.2, 24.816±0.2, 25.199±0.2, 25.980±0.2, 28.263±0.2, 31.299±0.2, 32.639±0.2, 33.540±0.2, and 34.978±0.2° 2θ using Cu Kα radiation.

In one embodiment, Form α is characterized by an XRPD pattern comprising peaks at approximately 7.8, 8.2, 10.2, 11.3, 11.9, 14.3, 14.7, 15.8, 16.2, 17.6, 18.4, 20.1, 20.5, 21.5, 22.7, 24.1, 24.8, 25.2, 26.0, 28.3, 31.3, 32.6, 33.5, 35.0, and 35.9° 2θ using Cu Kα radiation.

In one embodiment, Form α is characterized by an XRPD pattern comprising peaks at 7.780±0.2, 8.220±0.2, 10.218±0.2, 11.279±0.2, 11.907±0.2, 14.302±0.2, 14.721±0.2, 15.760±0.2, 16.218±0.2, 17.601±0.2, 18.402±0.2, 20.122±0.2, 20.500±0.2, 21.502±0.2, 22.719±0.2, 24.061±0.2, 24.816±0.2, 25.199±0.2, 25.980±0.2, 28.263±0.2, 31.299±0.2, 32.639±0.2, 33.540±0.2, 34.978±0.2, and 35.923±0.2° 2θ using Cu Kα radiation.

In one embodiment, Form α is characterized by an XRPD pattern comprising peaks at approximately the positions shown in the table below:

| Peak No. | 2-Theta | d-(A) | Intensity | L/L0 |
|---|---|---|---|---|
| 1 | 7.780 | 11.3542 | 217 | 31.2 |
| 2 | 8.220 | 10.7472 | 92 | 13.2 |
| 3 | 10.218 | 8.6498 | 66 | 9.5 |
| 4 | 11.279 | 7.8388 | 88 | 12.7 |
| 5 | 11.907 | 7.4266 | 29 | 4.2 |
| 6 | 14.302 | 6.1877 | 228 | 32.8 |
| 7 | 14.721 | 6.0124 | 190 | 27.3 |
| 8 | 15.760 | 5.6183 | 239 | 34.4 |
| 9 | 16.218 | 5.4608 | 371 | 53.4 |
| 10 | 17.601 | 5.0348 | 695 | 100 |
| 11 | 18.402 | 4.8172 | 43 | 6.2 |
| 12 | 20.122 | 4.4093 | 153 | 22 |
| 13 | 20.500 | 4.3288 | 551 | 79.3 |
| 14 | 21.502 | 4.1293 | 30 | 4.3 |
| 15 | 22.719 | 3.9108 | 37 | 5.3 |
| 16 | 24.061 | 3.6956 | 592 | 85.2 |
| 17 | 24.816 | 3.5849 | 71 | 10.2 |
| 18 | 25.199 | 3.5312 | 140 | 20.1 |
| 19 | 25.980 | 3.4268 | 509 | 73.2 |
| 20 | 28.263 | 3.155 | 165 | 23.7 |
| 21 | 31.299 | 2.8555 | 68 | 9.8 |
| 22 | 32.639 | 2.7413 | 155 | 22.3 |
| 23 | 33.540 | 2.6697 | 127 | 18.3 |
| 24 | 34.978 | 2.5631 | 50 | 7.2 |
| 25 | 35.923 | 2.4978 | 37 | 5.3 |

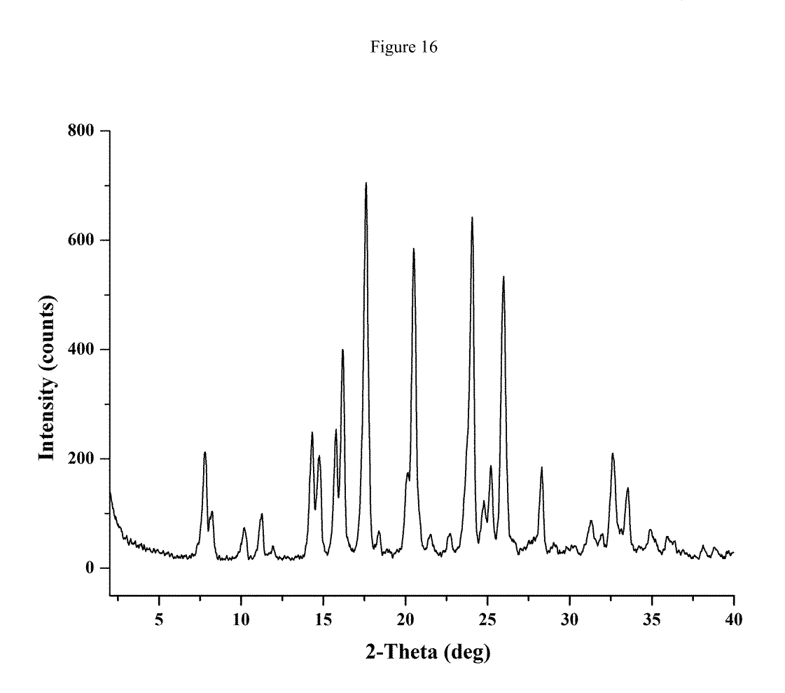

In one embodiment, Form α is characterized by an XRPD pattern substantially similar to that set forth in FIG. 16.

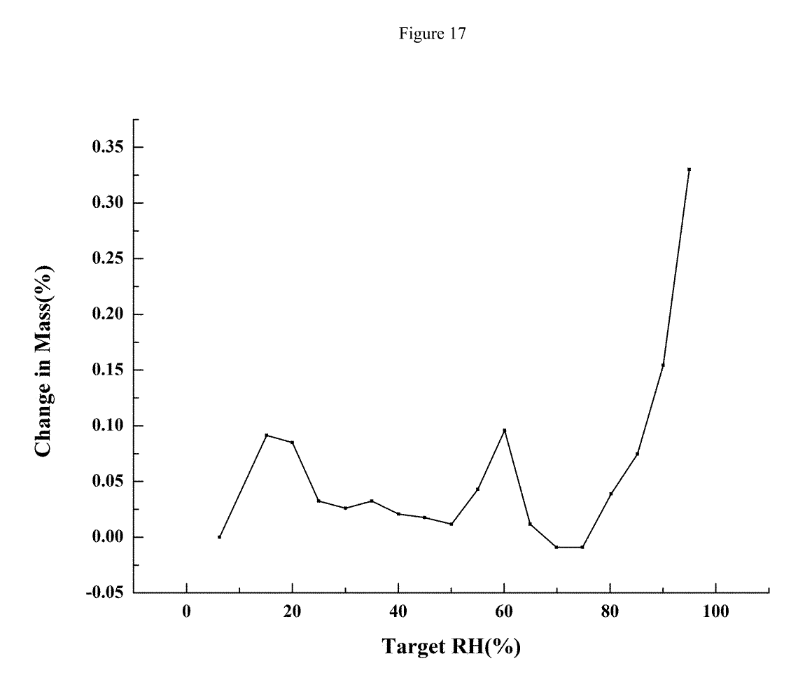

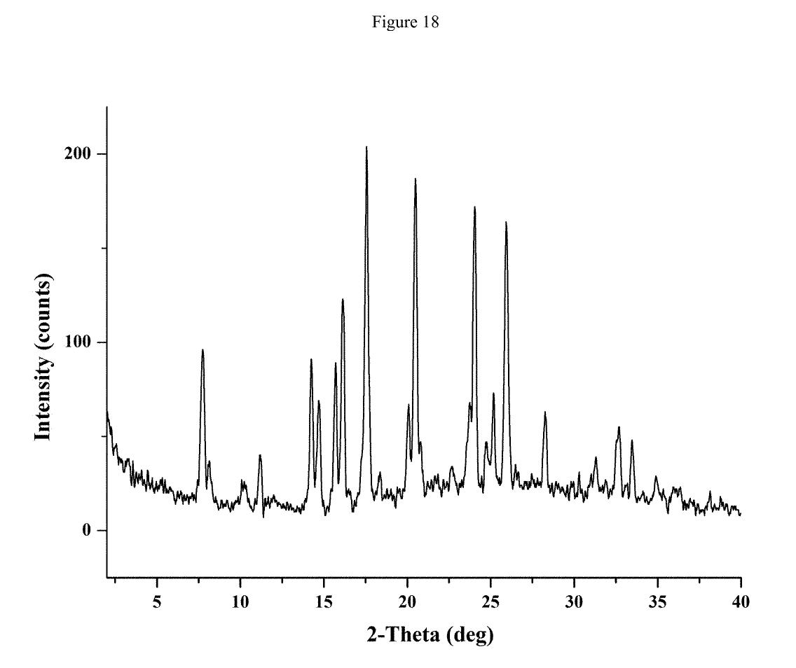

In one embodiment, Form α is stable under various humidity. In one embodiment, Form α displays low hygroscopicity under various humidity. In one embodiment, the water content of Form α does not change significantly (e.g., less than 0.01%, 0.02%, 0.03%, 0.04%, 0.05%, 0.06%, 0.07%, 0.08%, 0.09%, or 0.1%), upon change in humidity. In one embodiment, the water content of Form α does not change significantly at a humidity between 20% RH and 95% RH. In one embodiment, the change of the water content of Form α is as set forth in FIG. 17. In one embodiment, the XRPD pattern of Form α after exposure to various humidity (e.g., between 20% RH and 95% RH) is substantially the same as the XRPD pattern of Form α before exposure to humidity. In one embodiment, the XRPD pattern of Form α after exposure to various humidity (e.g., between 20% RH and 95% RH) is substantially similar to that set forth in FIG. 18.

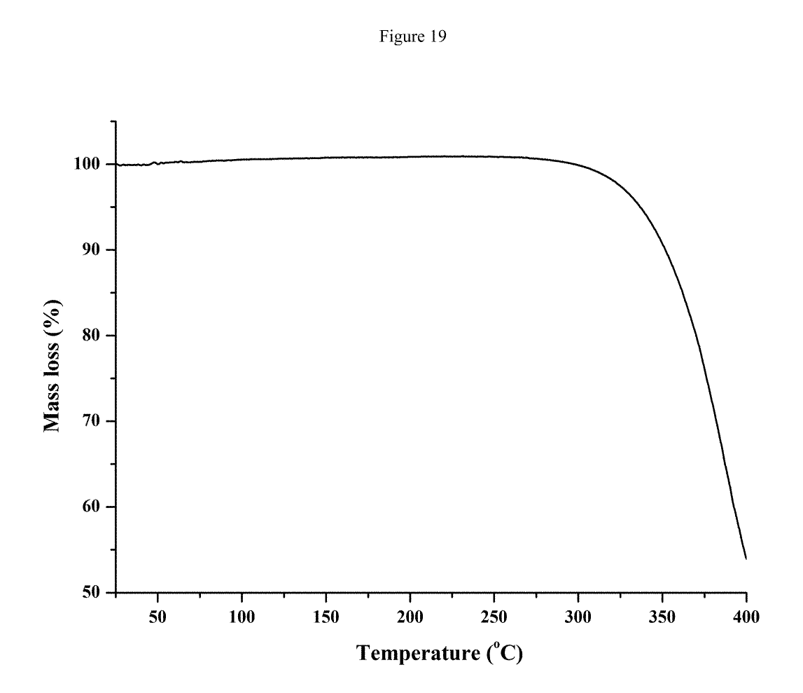

In one embodiment, Form α shows no weight loss when heated, as measured by TGA. In one embodiment, Form α is characterized by a TGA pattern substantially similar to that set forth in FIG. 19.

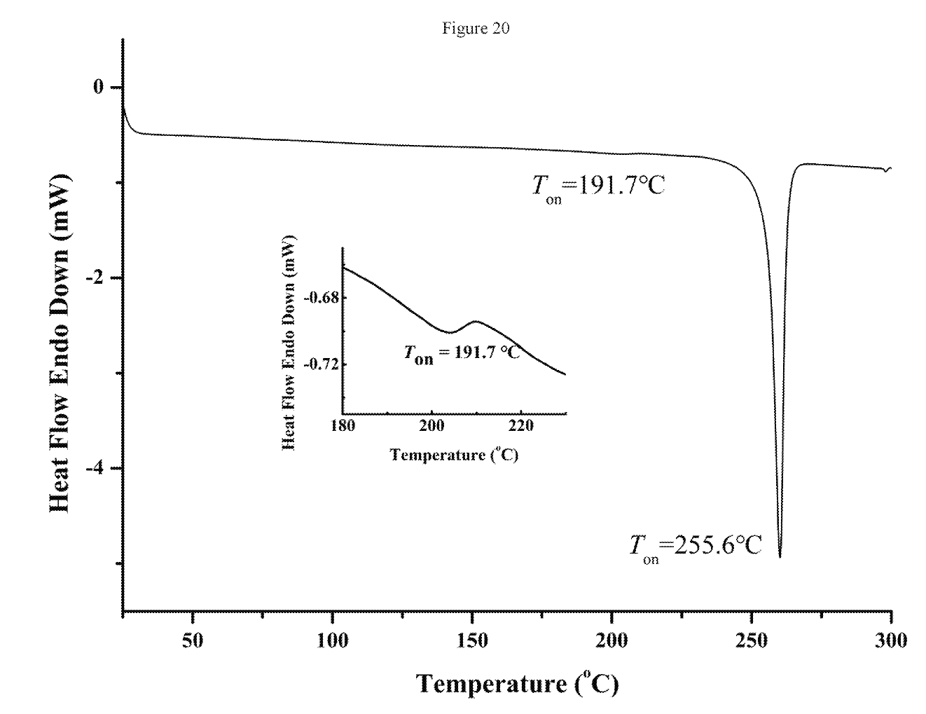

In one embodiment, Form α is characterized by an endothermic event with onset at approximately 192° C., as measured by DSC. In one embodiment, Form α is characterized by an endothermic event with onset at 191.7±5° C., as measured by DSC. In one embodiment, Form α is characterized by a melting event with onset at approximately 256° C., as measured by DSC. In one embodiment, Form α is characterized by a melting event with onset at 255.6±5° C., as measured by DSC. In one embodiment, Form α is characterized by a DSC thermogram substantially similar to that set forth in FIG. 20.

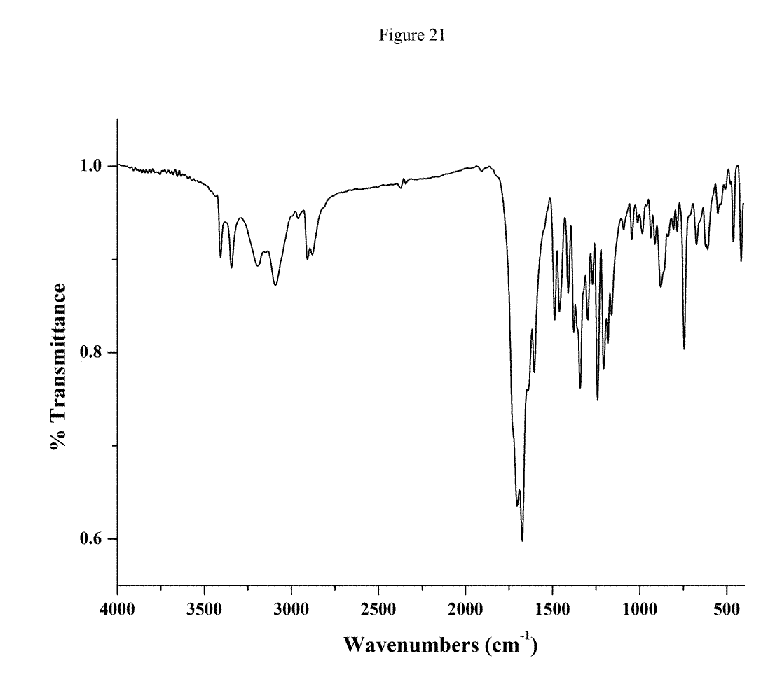

In one embodiment, Form α is characterized by an IR spectrum comprising peaks at approximately 3344, 3193, 3092, 1703, 1674, 1242, and 745 cm′. In one embodiment, Form α is characterized by an IR spectrum substantially similar to that set forth in FIG. 21.

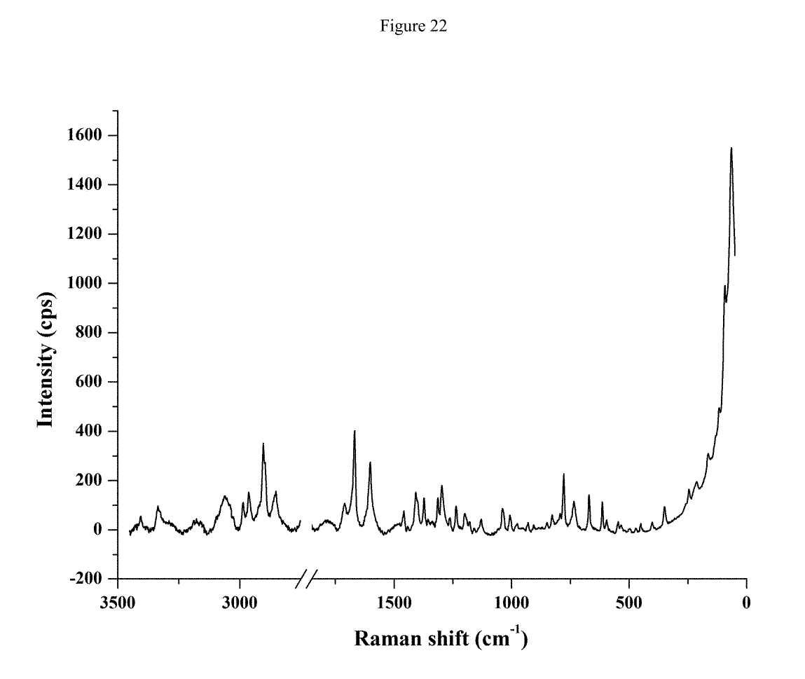

In one embodiment, Form α is characterized by a Raman spectrum comprising peaks at approximately 2964, 2904, 2858, 1668, 1601, 1408, 1297, 778, and 670 cm′. In one embodiment, Form α is characterized by a Raman spectrum substantially similar to that set forth in FIG. 22.

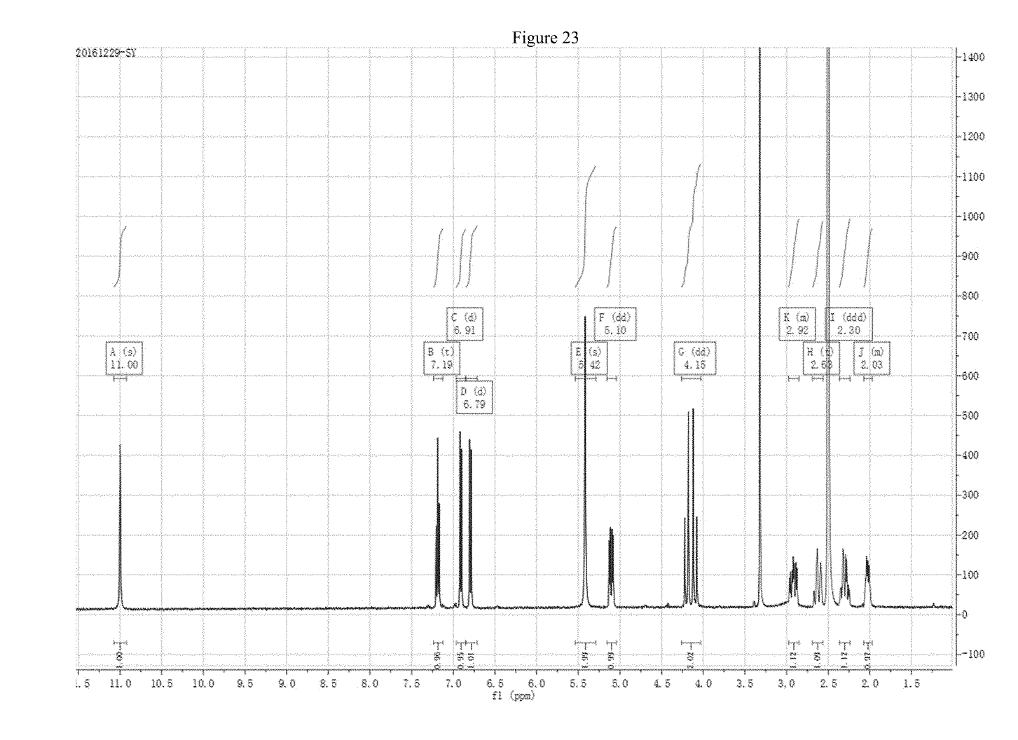

In one embodiment, Form α is characterized by a ¹H NMR spectrum as set forth below: ¹H NMR (DMSO-d₆) δ: 2.03 (m, 1H, CHCHₐCH_{b}CH₂CONH), 2.30 (ddd, 1H, CHCHₐCH_{b}CH₂CONH), 2.68 (t, 1H, CH₂CHₐCH_{b}CONH), 2.92 (m, 1H, CH₂CHₐCH_{b}CONH), 4.15 (dd, 2H, PhCH₂N), 5.10 (dd, 1H, NCHCO), 5.42 (s, 2H, PhNH₂), 6.79 (d, 1H, Ph), 6.91 (d, 1H, Ph), 7.19 (t, 1H, Ph), 11.00 (s, 1H, CONHCO). In one embodiment, Form α is characterized by a ¹H NMR spectrum substantially similar to that set forth in FIG. 23.

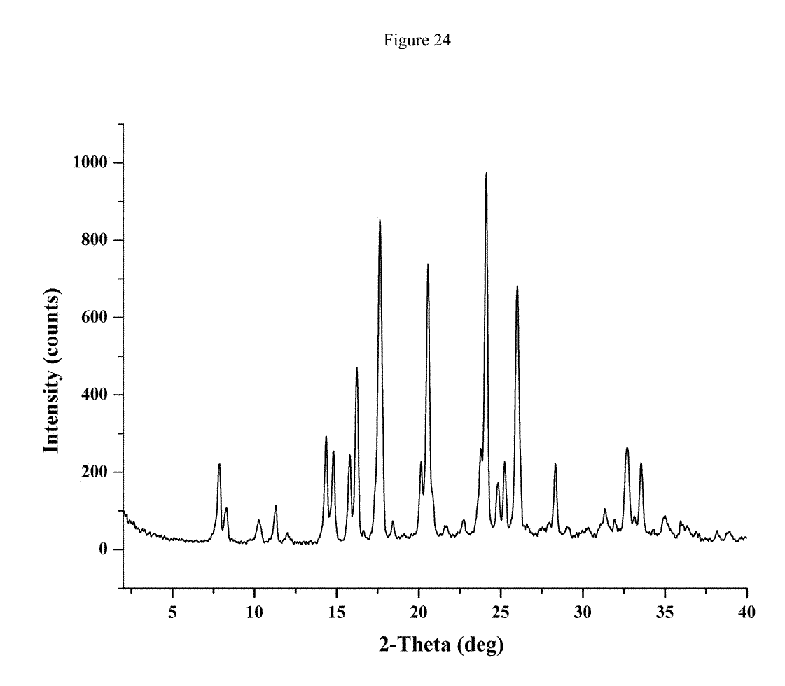

In one embodiment, Form α is stable upon mixing with a variety of solvents. In one embodiment, the solvent is selected from methanol, ethanol, acetone, acetonitrile, tetrahydrofuran, ethyl acetate, and dioxane. In one embodiment, upon mixing with methanol, Form α maintains its polymorphic form as Form α. In one embodiment, upon mixing with methanol, Form α is characterized by an XRPD pattern substantially similar to that set forth in FIG. 24.

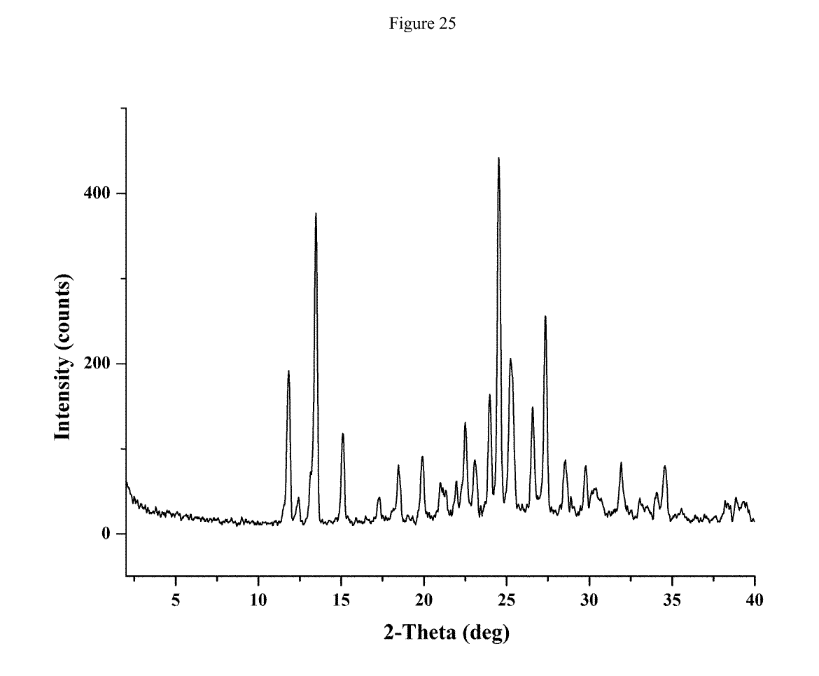

In one embodiment, upon mixing with water, Form α converts to Form DH. In one embodiment, upon mixing with water, Form α converts to a polymorph characterized by an XRPD pattern substantially similar to that set forth in FIG. 25.

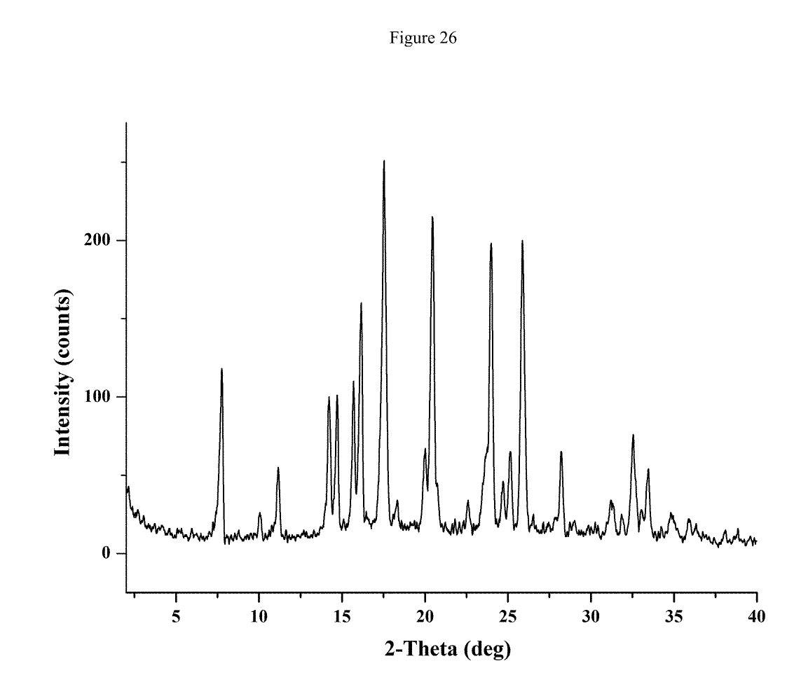

In one embodiment, Form α converts to a different polymorph at an elevated temperature (e.g., 230° C. or higher). In one embodiment, when heated to 230° C., Form α converts to a polymorph characterized by an XRPD pattern substantially similar to that set forth in FIG. 26 (“Form A”).

In one embodiment, Form α displays high stability (e.g., higher stability as compared to other polymorphs or crystal forms of Compound A) when Form α and the other polymorphs are subjected to substantially the same storage conditions (e.g., humidity and/or temperature).

In one embodiment, Form DH is stable under various storage conditions. In one embodiment, Form α is stable (e.g., the XRPD pattern of Form DH does not substantially change) after storage under 20° C./P₂O₅, 25° C./P₂O₅, 30° C./P₂O₅, 35° C./P₂O₅, or 40° C./P₂O₅, for at least a week, two weeks, three weeks, four weeks, six weeks, eight weeks, three months, four months, five months, six months, eight months, ten months, or twelve months. In one embodiment, Form α is stable after storage under 40° C./P₂O₅ for at least four weeks.

As used herein, the terms “polymorphs”, “polymorphic forms”, “crystalline polymorphs”, “crystal polymorphs”, and “crystal forms” and related terms refer to crystalline forms of the same molecule. Crystallization solvent, rate of crystallization, storage temperature, and other factors may cause one crystal form to dominate. Different polymorphs usually have different X-ray diffraction patterns, infrared spectra, and characteristics measured by other methods (e.g., DSC and TGA), and display different physical properties such as, for example, melting temperatures, heats of fusion, density, crystal shape, optical and electrical properties, solubilities, and dissolution rates as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Differences in stability can also result from changes in chemical reactivity (e.g., differential oxidation, such that a dosage form discolors more rapidly when comprised of one polymorph than when comprised of another polymorph) or mechanical property (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing, for example, one polymorph may be more likely to form solvates or might be difficult to filter and wash free of impurities (e.g., particle shape and size distribution might be different between polymorphs).

Once identified, polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion, and sublimation.

Techniques for characterizing polymorphs include, but are not limited to, differential scanning calorimetry (DSC), X-ray powder diffractometry (XRPD), single crystal X-ray diffractometry, vibrational spectroscopy (e.g., IR and Raman spectroscopy), TGA, DTA, DVS, solid state NMR, hot stage optical microscopy, scanning electron microscopy (SEM), electron crystallography and quantitative analysis, particle size analysis (PSA), surface area analysis, solubility studies, and dissolution studies.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures discussed in this application include all atropic isomers. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. All possible tautomeric forms of Compound A or a stereoisomer thereof are included within the scope of the present application. It is to be understood that the compound of the application may be depicted as different tautomers. Even though one tautomer may be described, the present application includes all tautomers.

In the present application, the structural formula of Compound A represents a certain stereoisomer for convenience in some cases, but the present application includes all stereoisomers, such as optical isomers based on an asymmetrical carbon, enantiomers, diastereomers, tautomers, and the like.

As used herein, the term “pure” means about 90-100%, particularly 95-100%, more particularly 98-100%, or 99-100% (wt./wt.) pure compound; e.g., less than about 10%, less than about 5%, less than about 2%, or less than about 1% impurity (wt./wt.) is present. Such impurities include, e.g., degradation products, oxidized products, solvents, and/or other undesirable impurities.

As used herein, a compound is “stable” where significant amounts of degradation products are not observed under constant conditions of humidity (e.g., 10%, 20%, 30%, 40%, 50%, 60%, 70%, 75%, 80%, 85%, 90%, and 95% RH), light exposure and temperatures (e.g., higher than 0° C., e.g., 20° C., 25° C., 30° C., 35° C., 40° C., 45° C., 50° C., 55° C., 60° C., 65° C., and 70° C.) over a certain period (e.g., one week, two weeks, three weeks, and four weeks). A compound is not considered to be stable at a certain condition when degradation impurities appear or an area percentage (e.g., AUC as characterized by HPLC) of existing impurities begins to grow. The amount of degradation growth as a function of time is important in determining compound stability.

As used herein, the term “mixing” means combining, blending, stirring, shaking, swirling, or agitating. The term “stirring” means mixing, shaking, agitating, or swirling. The term “agitating” means mixing, shaking, stirring, or swirling.

Unless explicitly indicated otherwise, the terms “approximately” and “about” are synonymous. In one embodiment, “approximately” and “about” refer to recited amount, value, or duration ±20%, ±15%, ±10%, ±8%, ±6%, ±5%, ±4%, ±2%, ±1%, or ±0.5%. In another embodiment, “approximately” and “about” refer to listed amount, value, or duration ±10%, ±8%, ±6%, ±5%, ±4%, or ±2%. In yet another embodiment, “approximately” and “about” refer to listed amount, value, or duration ±5%.

When the terms “approximately” and “about” are used when reciting XRPD peaks, these terms refer to the recited X-ray powder diffraction peak ±2.5° 2θ, ±2.0° 2θ, ±1.5° 2θ, ±1.0° 2θ, ±0.5° 2θ, ±0.3° 2θ, ±0.2° 2θ, or ±0.1° 2θ. In one embodiment, the terms “approximately” and “about” refer to the listed X-ray powder diffraction peak ±2.0° 2θ, ±1.5° 2θ, ±1.0° 2θ, ±0.5° 2θ, ±0.3° 2θ, ±0.2° 2θ, or ±0.1° 2θ. In one embodiment, the terms “approximately” and “about” refer to the listed X-ray powder diffraction peak ±1.0° 2θ, ±0.5° 2θ, ±0.3° 2θ, ±0.2° 2θ, or ±0.1° 2θ. In one embodiment, the terms “approximately” and “about” refer to the listed X-ray powder diffraction peak ±0.5° 2θ, ±0.3° 2θ, ±0.2° 2θ, or ±0.1° 2θ. In one embodiment, the terms “approximately” and “about” refer to the listed X-ray powder diffraction peak ±0.2° 2θ. In one embodiment, the terms “approximately” and “about” refer to the listed X-ray powder diffraction peak ±0.1° 2θ.

When the terms “approximately” and “about” are used when reciting temperature or temperature range, these terms refer to the recited temperature or temperature range ±5° C., ±2° C., or ±1° C. In another embodiment, the terms “approximately” and “about” refer to the recited temperature or temperature range ±2° C.

### Pharmaceutical Compositions

The present application also provides a pharmaceutical composition comprising a polymorph of the application, such as Form DH. A “pharmaceutical composition” is a formulation containing a polymorph of the present application in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of the active ingredient (e.g., a polymorph described herein) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration include powders, sprays, ointments, pastes, creams, lotions, gels, solutions, patches, and inhalants. In one embodiment, the active ingredient is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the application is formulated to be compatible with its intended route of administration. Examples of routes of administration include oral, parenteral (e.g., intravenous, intradermal, subcutaneous), intramuscular, topical, transdermal, and transmucosal administration. In one embodiment, the route of administration is oral.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon various factors, including but not limited to, subject's age, gender, weight, size, and health; the nature, extent, and severity of the condition; diet; time and frequency of administration; and the therapeutic or combination of therapeutics selected for administration. Therapeutically effective amounts for a given situation can be determined by routine experimentation that is within the skill and judgment of the clinician.

The therapeutically effective amount can be estimated initially either in cell culture assays, e.g., in cancer cells or animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred.

A pharmaceutical composition of the present application may be manufactured in a manner that is generally known, e.g., by means of conventional mixing, dissolving, granulating, dragee-making, levigating, emulsifying, encapsulating, entrapping, or lyophilizing processes. The pharmaceutical composition may be formulated in a conventional manner using one or more pharmaceutically acceptable carriers comprising excipients and/or auxiliaries that facilitate processing of the active ingredient into preparations that can be used pharmaceutically. The appropriate formulation is dependent upon the route of administration chosen.

Oral compositions generally include an inert diluent or an edible pharmaceutically acceptable carrier. They can be enclosed in gelatin capsules or compressed into tablets. For the purpose of oral administration, the active ingredient can be incorporated with excipients and used in the form of tablets, troches, or capsules. Oral compositions can also be prepared using a fluid carrier for use as a mouthwash, wherein the active ingredient in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Pharmaceutically compatible diluents include starch, dextrin, sucrose, glucose, lactose, mannitol, sorbitol, xylitol, microcrystalline cellulose, calcium sulfate, calcium hydrogen phosphate, calcium carbonate, and the like. Pharmaceutically compatible wetting agents included water, ethanol, isopropanol, and the like. Pharmaceutically compatible binders include starch pulp, dextrin, syrup, honey, glucose solution, microcrystalline cellulose, mucilage of arabic gum, gelatin mucilage, sodium hydroxymethylcellulose, methylcellulose, hydroxypropylmethylcellulose, ethyl cellulose, acrylic resin, carbomer, polyvinyl pyrrolidone, polyethylene glycol, and the like. Pharmaceutically compatible disintegrants include dry starch, microcrystalline cellulose, low-substituted hydroxypropylcellulose, cross-linked polyvinylpyrrolidone, croscarmellose sodium, sodium carboxymethyl starch, sodium bicarbonate and citric acid, polyoxyethylene sorbitol fatty acid esters, sodium dodecyl sulfonate and the like. Pharmaceutically compatible lubricants and glidants include talc powder, silica, stearate, tartaric acid, liquid paraffin, polyethylene glycol, and the like.

Pharmaceutical compositions suitable for injectable use (e.g., intravenous, intramuscular) include sterile aqueous solutions (where water soluble), dispersions/suspensions, and sterile powders for the extemporaneous preparation of sterile injectable solutions or dispersions. Suitable carriers include physiological saline, bacteriostatic water, Cremophor EL™ (BASF, Parsippany, N.J.) or phosphate buffered saline (PBS). The carriers can also be a solvent or dispersion medium containing, for example, water, ethanol, polyol (for example, glycerol, propylene glycol, and liquid polyethylene glycol, and the like), and suitable mixtures thereof. The composition must be sterile and should be fluid to the extent that easy syringeability exists. It must be stable under the conditions of manufacture and storage and must be preserved against contaminating by microorganisms such as bacteria and fungi. The proper fluidity can be maintained, for example, by the use of agents such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will include isotonic agents, for example, sugars, polyalcohols such as manitol, sorbitol, sodium chloride in the composition. Other excipients include, but are not limited to, antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Prolonged absorption of the injectable compositions can be brought about by including in the composition an agent which delays absorption, for example, aluminum monostearate and gelatin. The preparation can be enclosed in ampoules, disposable syringes or multiple dose vials made of glass or plastic.

Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

For administration by inhalation, the active ingredient is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active ingredient is formulated into ointments, salves, gels, or creams as generally known in the art.

The active ingredient can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

The pharmaceutical composition of the present application, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

Techniques for formulation and administration of the disclosed polymorph of the application can be found in Remington: the Science and Practice of Pharmacy, 19^{th} edition, Mack Publishing Co., Easton, Pa. (1995).

### Method of Preparing Polymorphs of the Application

### Form DH

The application also pertains, at least in part, to a method of preparing Form DH, comprising:

(1) dissolving Compound A in water to form a mixture;

(2) forming a slurry or suspension from the mixture from (1) at a temperature at or below 20° C.; and

(3) separating the solid from the slurry or suspension from (2) and drying the solid, such that Form H is formed.

In one embodiment, step (1) comprises sonication to dissolve Compound A. In one embodiment, a polymorph of Compound A is dissolved in step (1). In one embodiment, a polymorph of an anhydrate of Compound A is dissolved in step (1). In one embodiment, the Form A polymorph of Compound A as described in Chinese Patent No. 1871003 is dissolved in step (1). In one embodiment, step (1) comprises dissolving an excess amount of Compound A.

In one embodiment, the mixture from step (1) is cooled to approximately 20° C. to form a slurry or suspension. In one embodiment, the slurry or suspension is kept at approximately 20° C. for at least 24 hours. In one embodiment, the slurry or suspension is kept at approximately 20° C. for at least 48 hours. In one embodiment, the slurry or suspension is kept at approximately 20° C. for at least 72 hours. In one embodiment, the slurry or suspension is kept at approximately 20° C. for at least 96 hours. In one embodiment, the slurry or suspension is kept at approximately 20° C. for at least 108 hours.

In one embodiment, the mixture from step (1) is cooled to approximately 15° C. to form a slurry or suspension. In one embodiment, the slurry or suspension is kept at approximately 15° C. for at least 24 hours. In one embodiment, the slurry or suspension is kept at approximately 15° C. for at least 30 hours. In one embodiment, the slurry or suspension is kept at approximately 15° C. for at least 32 hours. In one embodiment, the slurry or suspension is kept at approximately 15° C. for at least 34 hours.

In one embodiment, the solid in the slurry or suspension is separated by removing the supernatant. In one embodiment, the solid is dried at approximately 40-60° C. In one embodiment, the solid is dried at approximately 40° C. In one embodiment, the solid is dried for approximately 10 hours. In one embodiment, the solid is dried at approximately 40° C. for approximately 10 hours.

In one embodiment, the method optionally comprises, before step (3), centrifuging the slurry or suspension from (2).

Form α

The application also pertains, at least in part, to a method of preparing Form α, comprising

(1) dissolving Compound A in nitromethane to form a mixture;

(2) forming a slurry or suspension from the mixture from (1) at a temperature of approximately 20° C.; and

(3) separating the solid from the slurry or suspension from (2) and drying the solid, such that Form α is formed.

In one embodiment, step (1) comprises sonication to dissolve Compound A. In one embodiment, a polymorph of Compound A is dissolved in step (1). In one embodiment, a polymorph of an anhydrate of Compound A is dissolved in step (1). In one embodiment, the Form A polymorph of Compound A as described in Chinese Patent No. 1871003 is dissolved in step (1). In one embodiment, step (1) comprises dissolving an excess amount of Compound A.

In one embodiment, the mixture from step (1) is cooled to approximately 20° C. to form a slurry or suspension. In one embodiment, the slurry or suspension is kept at approximately 20° C. for at least 24 hours.

In one embodiment, the solid in the slurry or suspension is separated by removing the supernatant. In one embodiment, the solid is dried at approximately 60-70° C. In one embodiment, the solid is dried for approximately 7 hours. In one embodiment, the solid is dried at approximately 60-70° C. for approximately 7 hours.

In one embodiment, the method optionally comprises, before step (3), centrifuging the slurry or suspension from (2).

In one embodiment, the method optionally comprises, after step (3), step (4) washing the Form α from step (3) with methanol or ethanol, separating, and drying the Form α.

### Methods of Treatment

The application also pertains, at least in part, to a method of treating or preventing a disease or condition in a subject in need thereof, comprising administering to the subject a therapeutically effective amount of a polymorph of the present application.

The application also pertains, at least in part, to a polymorph of the present application for treating or preventing a disease or condition.

The application also pertains, at least in part, to use of a polymorph of the present application in the manufacture of a medicament for treatment or prevention of a disease or condition.

In one embodiment, the disease or condition is a cell proliferative disorder. The cell proliferative disorder can be cancer or a precancerous condition.

In one embodiment, the cell proliferative disorder is cancer.

In one embodiment, the cancer is a blood cancer. In one embodiment, the blood cancer is multiple myeloma, non-Hodgkin's lymphoma, Hodgkin's lymphoma, chronic lymphocytic leukemia, chronic myelogenous leukemia, acute lymphoblastic leukemia, acute myelogenous leukemia, or mantle cell lymphoma. In one embodiment, the cell proliferative disorder is multiple myeloma, chronic lymphocytic leukemia, or mantle cell lymphoma. In one embodiment, the cell proliferative disorder is multiple myeloma.

In one embodiment, the cancer is a solid tumor.

In one embodiment, the cell proliferative disorder is a non-cancerous condition, such as myelodysplastic syndromes.

In one embodiment, the disease or condition is inflammation.

In one embodiment, the disease or condition is autoimmune diseases.

As used herein, a “subject in need thereof” is a subject having a disease or condition against which a polymorph of the application is effective (e.g., cancer), or a subject having an increased risk of developing a disease or condition against which a polymorph of the application is effective (e.g., cancer) relative to the population at large. A “subject” includes a mammal. The mammal can be e.g., any mammal, e.g., a human, primate, bird, mouse, rat, fowl, dog, cat, cow, horse, goat, camel, sheep or a pig. Particularly, the mammal is a human.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue.

A cell proliferative disorder includes a precancer or a precancerous condition. A cell proliferative disorder includes cancer. Particularly, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin's lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin's lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

As used herein, “treating” or “treat” describes the management and care of a patient for the purpose of combating a disease, condition, or disorder and includes the administration of a polymorph of the present application, to alleviate the symptoms or complications of the disease, condition or disorder, or to eliminate the disease, condition or disorder.

As used herein, “preventing” or “prevent” describes reducing or eliminating the onset of the symptoms or complications of a disease, condition or disorder.

As used herein, the term “alleviate” is meant to describe a process by which the severity of a sign or symptom of a disease, condition or disorder is decreased. Importantly, a sign or symptom can be alleviated without being eliminated. In one embodiment, the administration of a polymorph of the application leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

Treating cancer can result in a reduction in size of a tumor. A reduction in size of a tumor may also be referred to as “tumor regression”. Treating cancer can result in a reduction in tumor volume, a decrease in the number of tumors and/or metastatic lesions in other tissues or organs distant from the primary tumor site, an increase in average survival time and/or a decrease in the mortality rate of a population of treated subjects in comparison to a population receiving carrier alone, to a population of untreated subjects, or to a population receiving monotherapy with a drug that is not a polymorph of the present application. Treating cancer can result in a decrease in tumor growth rate.

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present disclosure the skilled artisan can identify and employ other components and methodology useful for practicing the present application.

### EXAMPLES

### Example 1: Preparation of Form DH

30 mg Compound A was placed in a 4 mL container. After 2 mL water was added, the container was sonicated to dissolve Compound A. The resulting supersaturated suspension was kept at 20° C. for 24 hours. After centrifugation, the supernatant was removed, and the solid was dried at 40° C. in a drying oven for 10 hours, and Form DH was obtained (FIG. 1).

### Example 2: Study of Polymorphic Forms of Compound A

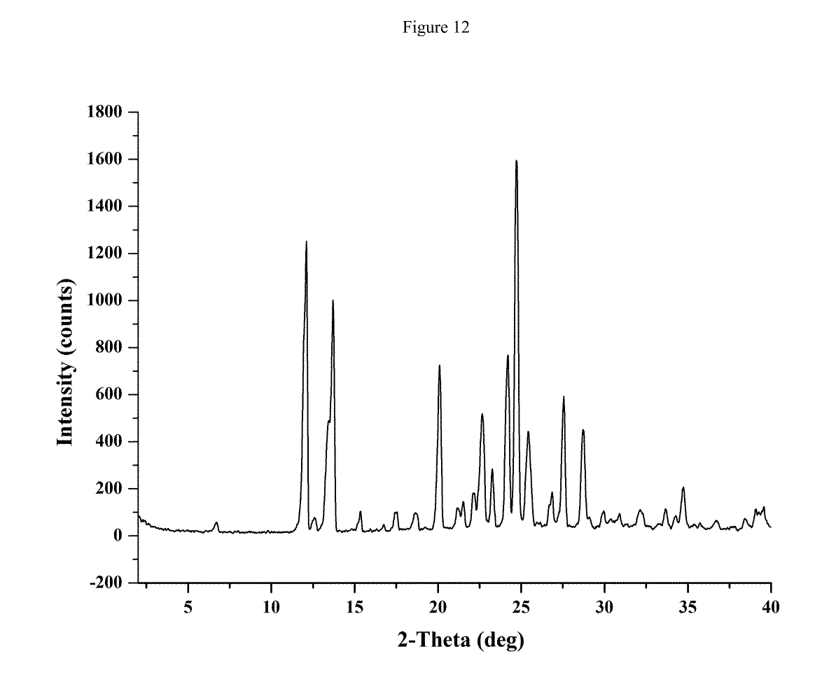

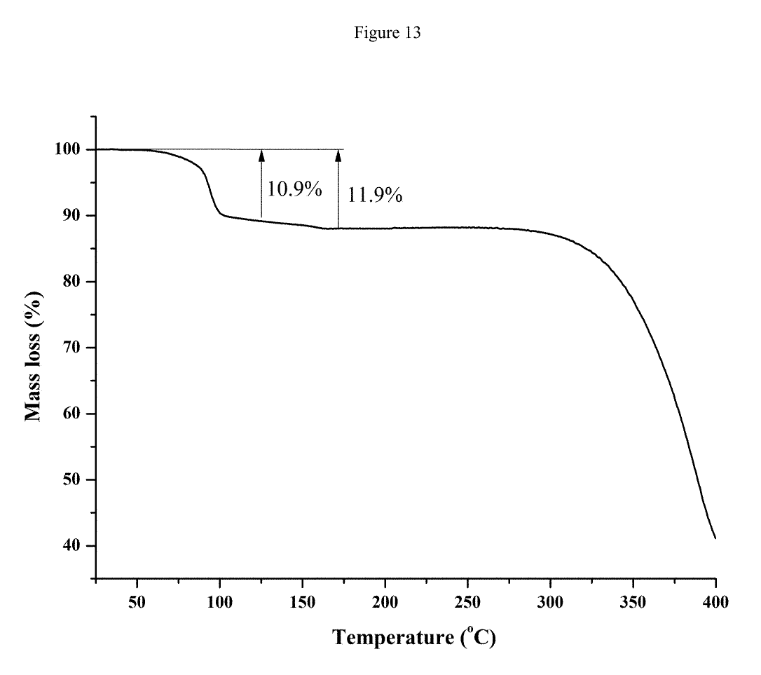

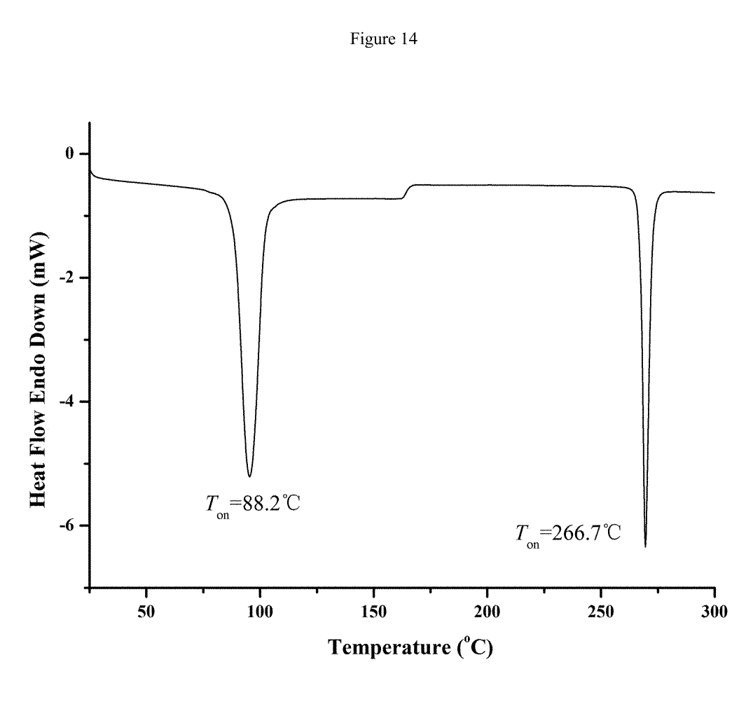

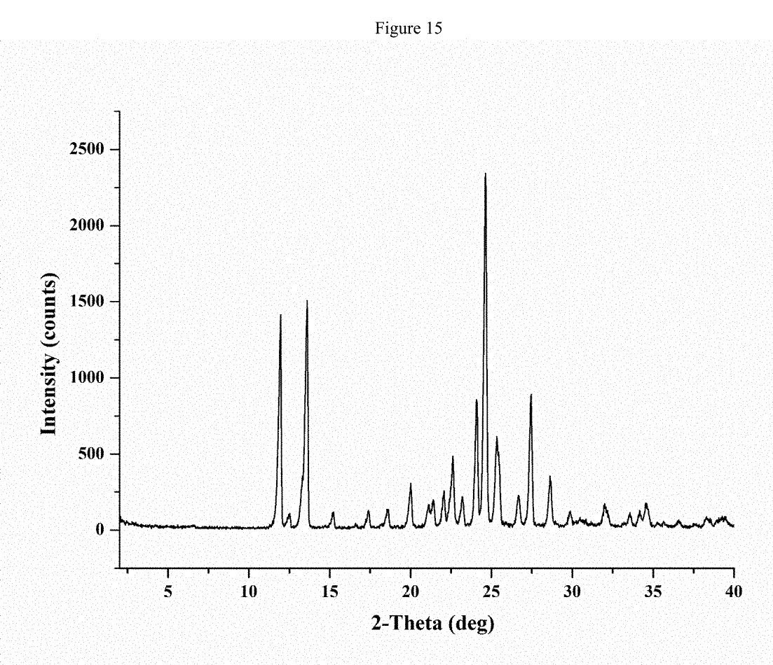

600 mg Compound A was placed in a 100 mL container. After 40 mL water was added, the container was stirred at 300 rpm at 20° C. After 12 hours, a sample was taken from the container and an XRPD was measured. The result showed that the XRPD was the same as the XRPD of the Form E polymorph described in Chinese Patent No. 1871003. The XRPD, TG, and DSC of Form E are shown in FIGS. 12, 13, and 14, respectively. However, after the sample was stirred at 20° C. for 108 hours, Form DH was formed, as demonstrated by the XRPD (FIG. 15).

### Example 3: DVS Study of Form DH

About 3 mg Form DH prepared in Example 1 was subject to dynamic vapor sorption analysis with a VTI-SA+ type dynamic vapor sorption analyzer (US TA Instruments Company). The temperature was 25° C., and the range of the relative humidity was 1-95%. No weight increase in the sample Form DH was observed under the test conditions, as shown in FIG. 2. Moreover, the crystal form remained unchanged (FIG. 3).

### Example 4: Preparation of Form α

30 mg Compound A was placed in a 4 mL container. After 2 mL nitromethane was added, the container was sonicated to dissolve Compound A. The resulting supersaturated suspension was kept at 20° C. for 24 hours. After centrifugation, the supernatant was removed, and the solid was dried at 60° C. in a drying oven for 7 hours, and a polymorph (i.e., Form α) of Compound A was obtained (FIG. 16). The Form α polymorph may be washed with methanol to yield the Form α polymorph as pale solid.

### Example 5: DVS Study of Form α

About 3 mg Form α prepared in Example 4 was subject to Dynamic Vapor Sorption analysis with a VTI-SA+ type dynamic vapor sorption analyzer (US TA Instruments Company). The temperature was 25° C., and the range of the relative humidity was 1-95%. No weight increase in the sample Form DH was observed under the test conditions, as shown in FIG. 17. Moreover, the crystal form remained unchanged (FIG. 18).

### Example 6: Study of Polymorphic Forms of Compound A

600 mg Compound A was placed in a 100 mL container. After 40 mL water was added, the container was stirred at 300 rpm at 25° C. After 48 hours, a sample was taken from the container and an XRPD was measured. The result showed that the XRPD was the same as the XRPD of the Form E polymorph described in Chinese Patent No. 1871003. A sample taken after 168 hours at 25° C. still had the same XRPD as the Form E polymorph described in Chinese Patent No. 1871003.

600 mg Compound A was placed in a 100 mL container. After 40 mL water was added, the container was stirred at 300 rpm at 15° C. After 10 hours, a sample was taken from the container and an XRPD was measured. The result showed that the XRPD was the same as the XRPD of the Form E polymorph described in Chinese Patent No. 1871003. However, after the sample was stirred at 15° C. for 34 hours, Form DH was formed, as demonstrated by the XRPD.

### Example 7: X-Ray Powder Diffraction (XRPD)

XRPD patterns of the polymorphs obtained in the Examples were collected on a D/MAX 2500 diffractometer using the following setting: Cu Kα radiation λ 1.5418 Å, 40 kV, 200 mA, 20 range 2-40° at 8°/min. The results are shown in the Figures.

### Example 8: Thermo-Gravimetric Analysis (TGA)

TGA data of the polymorphs obtained in the Examples were collected on a Mettler TGA/DSC1 analyzer. The measurement was conducted under nitrogen, and the samples were heated at 10° C./min. The results are shown in the Figures.

### Example 9: Differential Scanning Calorimetry (DSC)

DSC data of the polymorphs obtained in the Examples were collected on a Mettler DSC1 analyzer. The measurement was conducted under nitrogen, and the samples were heated at 10° C./min. The results are shown in the Figures.

### Example 10: IR and Raman Spectra

The IR and Raman spectra of the polymorphs obtained in the Examples were measured on TENSOR 27 IR spectrometer at room temperature in the range of 4000-400 cm⁻¹ and DXR Raman spectrometer at room temperature in the range of 3450-50 cm⁻¹. The results are shown in the Figures.

### Example 11: Stability of Form DH and Form α

15 mg of the polymorph obtained in Example 1 was placed in a 2 mL container. 1 mL of the each of the following solvents was added: methanol, ethanol, acetone, acetonitrile, tetrahydrofuran, nitromethane, ethyl acetate, or dioxane. The container was sonicated to dissolve Compound A. The resulting supersaturated suspension was kept at 20° C. for 24 hours. After centrifugation, the supernatant was removed, and the solid was dried at 45° C. for 2 hours. The XRPD of the solid was measured. Form DH converted to Form C polymorph in acetone (FIG. 9), but maintained its polymorphic form as Form DH in water, methanol (FIG. 8), ethanol, acetonitrile, tetrahydrofuran, ethyl acetate, and dioxane.

15 mg of the polymorph obtained in Example 4 was placed in a 2 mL container. 1 mL of the each of the following solvents was added: methanol, ethanol, acetone, acetonitrile, tetrahydrofuran, ethyl acetate, dioxane, or water. The container was sonicated to dissolve Compound A. The resulting supersaturated suspension was kept at 20° C. for 24 hours. After centrifugation, the supernatant was removed, and the solid was dried at 45° C. for 2 hours. The XRPD of the solid was measured. Form α converted to Form DH (FIG. 25) in water, but maintained its polymorphic form as Form α in methanol (FIG. 24), ethanol, acetone, acetonitrile, tetrahydrofuran, ethyl acetate, and dioxane.

Comparison of Form DH and Form α with other polymorphic forms of Compound A is shown in the Table below.

| | DH | α | A | B | E |
|---|---|---|---|---|---|
| Polymorph | Dihydrate | Anhydrous | Anhydrous | Hemihydrate | Dihydrate |
| Crystallinity | Good | Good | Good | Good | Good |
| Stability in | Cv. to | Cv. to | Cv. to | Cv. to Form A in | Cv. to Form |
| solution | Form C in | Form DH | Form B in | THF, to Form C | C in acetone, |
| | acetone | in water | water, to | in acetone, | to Form F in |
| | | | Form C in | may Cv. to Form | THF |
| | | | acetone | E in the presence | |
| | | | | of water | |
| Thermal | Cv. to | Cv. to | No Cv. | Cv. to Form A at | Cv. to Form |
| stability | Form F at | Form A at | | 175° C. | B at 125° C., |
| | 170° C. | 230° C. | | | to Form F at |
| | | | | | 175° C. |
| Accelerated | Stable | Stable after | Stable for | Stable for 85 | Stable after |
| stability | after 28 | 28 days at | 85 days at | days at | 28 days at |
| | days at | RT/P₂O₅, | RT/0% RH, | RT/0% RH, or | 40° C./75% |
| | 40° C./75% | Cv. to | or | 40° C./93% RH | RH, Cv. to |
| | RH, | Form A at | 40° C./93% | | Form H at |
| | Cv. to | 40° C./75% | RH | | RT/P₂O₅ |
| | Form E at | RH after 28 | | | after 28 days |
| | RT/P₂O₅ | days | | | |
| | after 28 | | | | |
| | days | | | | |
| Hygroscopicity | No significant | No significant | No significant | No significant | No |
| | Wt. | Wt. | Wt. | Wt. increase at | significant |
| | increase at | increase at | increase at | 5~95% RH | Wt. increase |
| | 1~95% RH | 1~95% RH | 5~95% RH | | at 5~95% RH |
| | No change | No change | | | |
| | in crystal | in crystal | | | |
| | form after | form after | | | |
| | DVS | DVS | | | |
| Dissolution | No crystal | Cv. to | Cv. to | N.A. | No crystal |
| | Cv. | Form E | Form E | | Cv. |

| | C | D | F | G | H |
|---|---|---|---|---|---|
| Polymorph | Acetone | Solvated, | Anhydrous | Anhydrous | 0.25/0.26 |
| | solvate | including | | | moles of |
| | | water and | | | crystallization |
| | | acetonitrile | | | water |
| Crystallinity | Good | Good | Obtained by | Obtained by | Obtained by |
| | | | dehydration | slurrying | storing Form |
| | | | of Form E, | Forms B and | E at RT and |
| | | | relatively | E in THF, | 0% RH for 7 |
| | | | poor | relatively | days, |
| | | | crystallinity | poor | relatively poor |
| | | | | crystallinity | crystallinity |
| Stability in | Cv. to Form | Cv. to Form | N.A. | N.A. | Cv. to Form A |
| solution | A in THF, to | A in THF, | | | in THF, to |
| | Form E in | to Form E in | | | Form E in |
| | water | water, to | | | water, to |
| | | Form C in | | | Form C in |
| | | acetone | | | acetone |
| Thermal | Cv. to Form | Cv. to Form | N.A. | N.A. | N.A. |
| stability | A at 150° C. | A at 150° C. | | | |
| | by | by | | | |
| | desolvation | desolvation | | | |
| Accelerated | Cv. to Form | Cv. to Form | N.A. | N.A. | N.A. |
| stability | B when | B when | | | |
| | stored at | stored at | | | |
| | 84% RH for | 84% RH for | | | |
| | 10 days | 10 days | | | |
| Hygroscopicity | No | No | N.A. | N.A. | N.A. |
| | significant | significant | | | |
| | Wt. increase | Wt. increase | | | |
| | at 5~85% | at 5~95% | | | |
| | RH, Wt. loss | RH | | | |
| | by 6.03% at | | | | |
| | 95% RH | | | | |
| Dissolution | N.A. | N.A. | N.A. | N.A. | N.A. |

### Example 12: Pharmaceutical Composition of Form DH

A pharmaceutical composition of Form DH was prepared according to the table below.

| Form | | | Microcrystalline | Polyvinyl | Magnesium |
|---|---|---|---|---|---|
| DH | Starch | water | cellulose | pyrrolidone | stearate |
| 20 g | 20 g | as | 2 g | 2 g | 1 g |
| | | appro- | | | |
| | | priate | | | |

Form DH and starch were mixed, followed by addition of microcrystalline cellulose and water. After the resulting mixture was passed through a 20-mesh sieve, and then a 18-mesh sieve, polyvinylpyrrolidone and magnesium stearate were mixed in to make 100 capsules.

### Example 13: Pharmaceutical Composition of Form DH

A pharmaceutical composition of Form DH was prepared according to the table below.

| Form | | | Microcrystalline | Polyvinyl | Magnesium |
|---|---|---|---|---|---|
| α | Starch | water | cellulose | pyrrolidone | stearate |
| 20 g | 20 g | as | 2 g | 2 g | 1 g |
| | | appro- | | | |
| | | priate | | | |

Form α polymorph and starch were mixed, followed by addition of microcrystalline cellulose and water. After the resulting mixture was passed through a 20-mesh sieve, and then a 18-mesh sieve, polyvinylpyrrolidone and magnesium stearate were mixed in to make 100 capsules.

### Example 14: Accelerated Stability Studies of Form DH and Form α

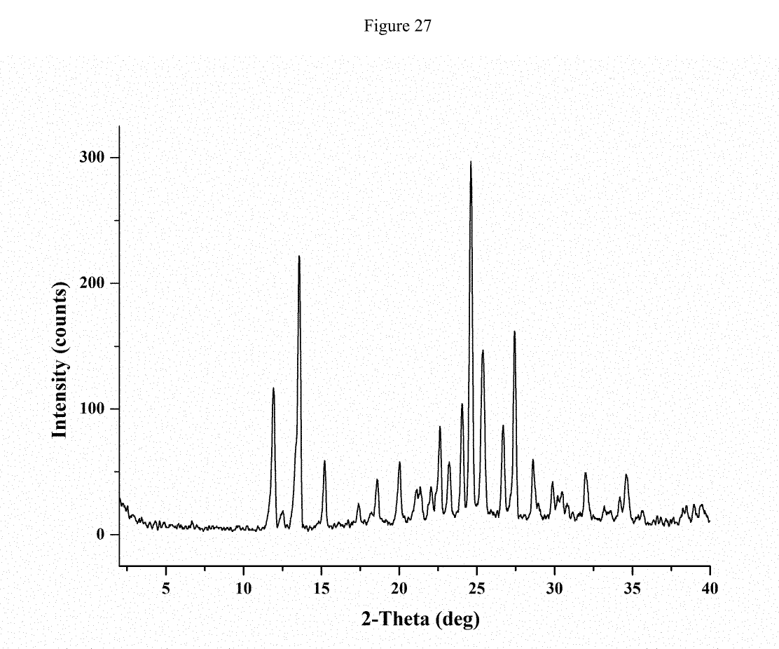

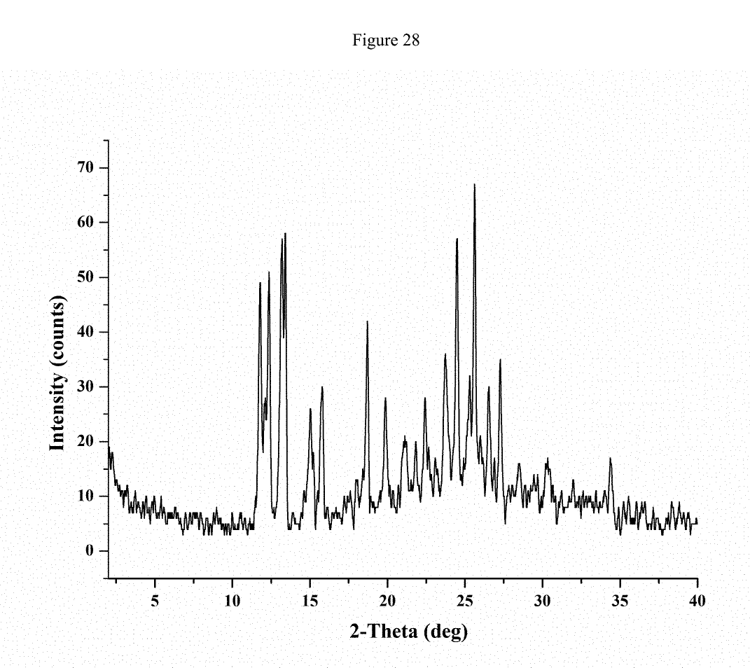

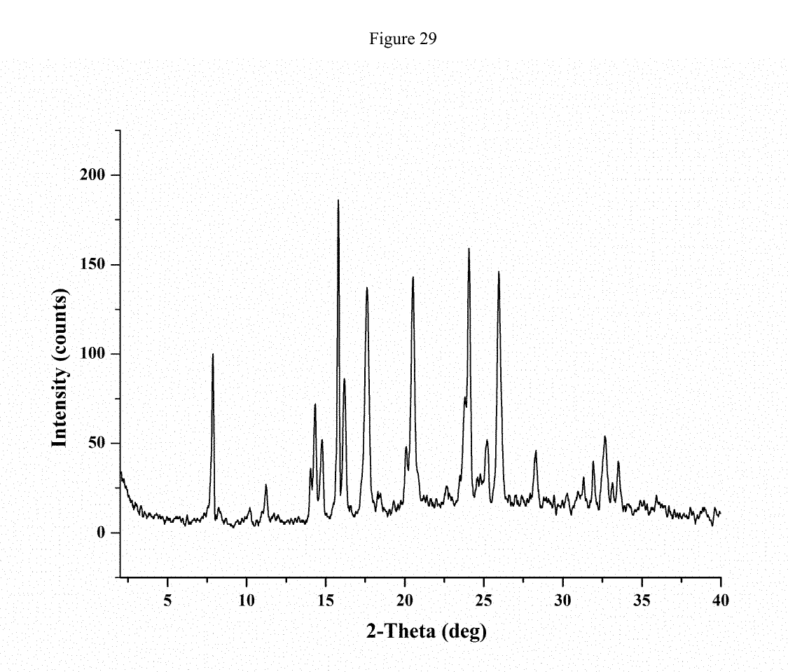

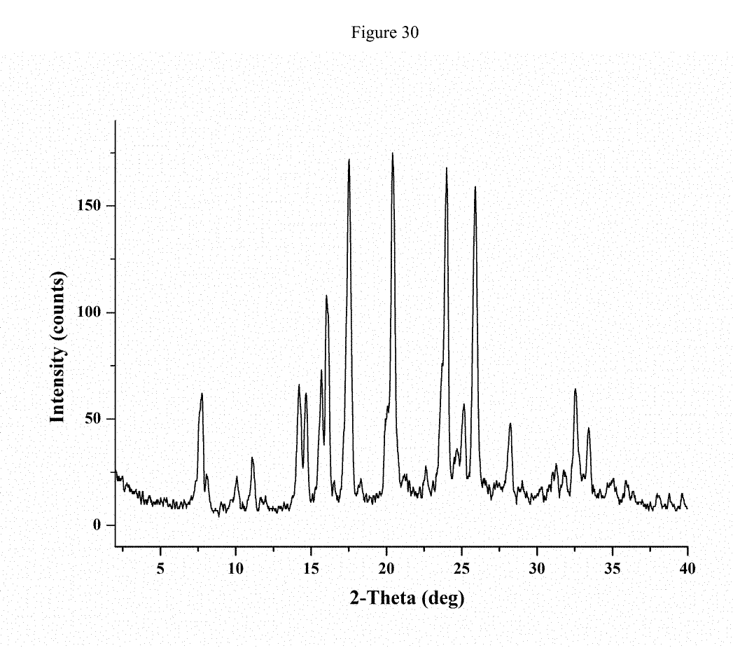

10 mg of Form DH or Form α of the present application was each put in a testing tube. The testing tubes were placed separately in sealed storage bags. The bags were stored under 40° C./75% RH or RT/P₂O₅. Samples of the polymorph were collected at day 1, day 3, day 5, day 7, day 14, day 21, and day 28 for XRPD measurement. As shown in FIGS. 27-30, the XRPD of Form H did not change after 28 days at 40° C./75% RH, and the XRPD of Form α did not change after 28 days at RT/P₂O₅.

### Example 15: Powder Dissolution Studies of Various Polymorphs of Compound A

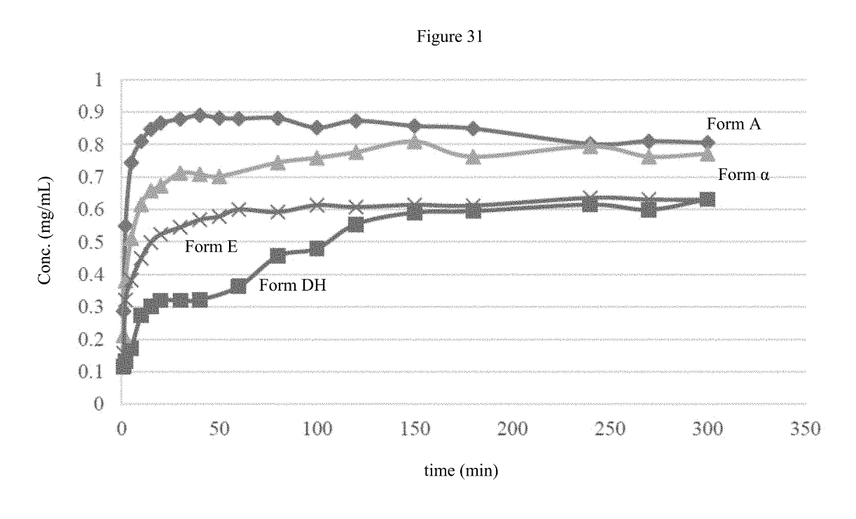

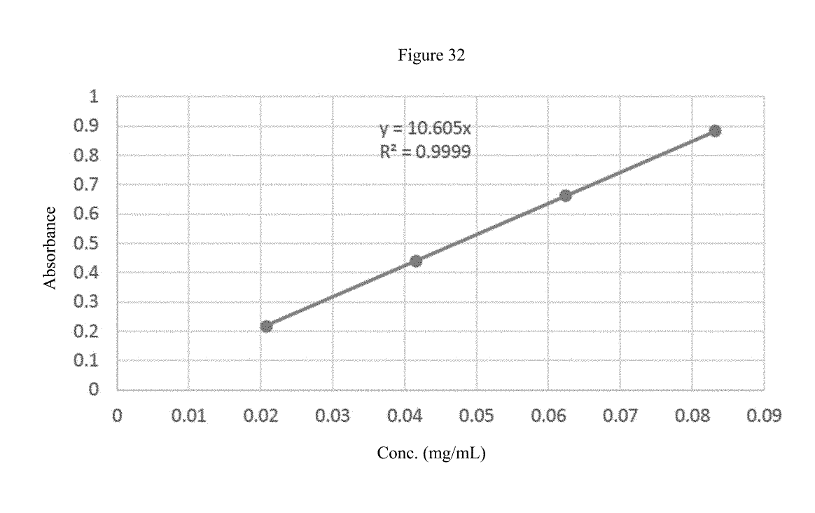

500 mg of Form DH or Form α of the present application, and the Form A or Form E polymorphs according to CN1871003 was each mixed with 500 mL water, and then placed in a RC-6 instrument, operated at 50 rpm with a 100-mesh sieve. Samples (2 mL) were collected at 1 min, 2 min, 5 min, 10 min, 15 min, 20 min, 30 min, 40 min, 50 min, 60 min, 80 min, 100 min, 120 min, 150 min, 180 min, 210 min, 240 min, 270 min, and 300 min. Water (2 mL) was added each time when a sample was taken. The samples were filtered through 0.22 μm film, diluted, and quantified according to a standard UV curve (shown in FIG. 32). The XRPD of the solid samples were measured. As shown in FIG. 31, Form DH did not change during the study.

1. A polymorph of a dihydrate of Compound A: 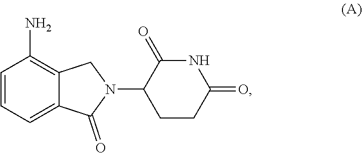 [SMILES: Nc1cccc2c1CN(C1CCC(=O)NC1=O)C2=O]
or a stereoisomer thereof, characterized by an X-ray powder diffraction (XRPD) pattern comprising peaks at approximately 12.0, 13.6, and 24.7° 2θ using Cu Kα radiation.

2. The polymorph of claim 1, characterized by an XRPD pattern comprising peaks at approximately 12.0, 13.6, 24.7, and 27.5° 2θ using Cu Kα radiation.

4. The polymorph of claim 1, characterized by an XRPD pattern comprising peaks at approximately 12.0, 13.6, 24.1, 24.7, 25.4, and 27.5° 2θ using Cu Kα radiation.

5. The polymorph of claim 1, characterized by an XRPD pattern substantially similar to that set forth in FIG. 1.

6. A polymorph of of Compound A anhydrate: 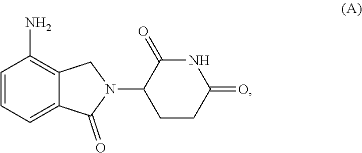 [SMILES: Nc1cccc2c1CN(C1CCC(=O)NC1=O)C2=O]
or a stereoisomer thereof, characterized by an XRPD pattern comprising peaks at approximately 17.6, 20.5, and 24.1° 2θ using Cu Kα radiation.

7. The polymorph of claim 6, characterized by an XRPD pattern comprising peaks at approximately 17.6, 20.5, 24.1, and 26.0° 2θ using Cu Kα radiation.

8. The polymorph of claim 6, characterized by an XRPD pattern comprising peaks at approximately 16.2, 17.6, 20.5, 24.1, and 26.0° 2θ using Cu Kα radiation.

9. The polymorph of claim 6, characterized by an XRPD pattern comprising peaks at approximately 7.8, 14.3, 15.8, 16.2, 17.6, 20.5, 24.1, and 26.0° 2θ using Cu Kα radiation.

10. The polymorph of claim 6, characterized by an XRPD pattern substantially similar to that set forth in FIG. 16.

11. A method of preparing the polymorph of claim 1, comprising
(1) dissolving Compound A in water to form a mixture;
(2) forming a slurry or suspension from the mixture from (1) at a temperature at or below 20° C.; and
(3) separating the solid from the slurry or suspension from (2) and drying the solid, such that Form H is formed.

12. A method of preparing the polymorph of claim 6, comprising
(1) dissolving Compound A in nitromethane to form a mixture;
(2) forming a slurry or suspension from the mixture from (1) at a temperature of approximately 20° C.; and
(3) separating the solid from the slurry or suspension from (2) and drying the solid, such that Form α is formed.

13. The method of claim 12, further comprising (4) washing the Form α from step (3) with methanol or ethanol, separating, and drying the Form α.

14. A pharmaceutical composition comprising the polymorph of claim 1, and a pharmaceutically acceptable diluent, excipient or carrier.

15. A pharmaceutical composition comprising the polymorph of claim 6, and a pharmaceutically acceptable diluent, excipient or carrier.

16. A method of treating or preventing disease or condition in a subject in need thereof, comprising administering to the subject a therapeutically effective amount of the polymorph of claim 1.

17. The method of claim 16, wherein the cancer is a blood cancer.

18. A method of treating or preventing disease or condition in a subject in need thereof, comprising administering to the subject a therapeutically effective amount of the polymorph of claim 6.

19. The method of claim 18, wherein the cancer is a blood cancer.
